(19) United States
(12) Patent Application Publication
(10) Pub. No.: US 2026/0306616 A1
(43) Pub. Date: Oct. 8, 2026

(54) Spike Protein Based Genetic Vaccines

(71) Applicant: University of Maryland, Baltimore, Baltimore, MD (US)

(72) Inventors: Syed Saif Hasan, Frederick, MD (US); Suruchi Singh, Rockville, MD (US)

(73) Assignee: University of Maryland, Baltimore, Baltimore, MD (US)

(21) Appl. No.: 19/638,377

(22) Filed: Apr. 3, 2026

Related U.S. Application Data
(60) Provisional application No. 63/783,863, filed on Apr. 4, 2025.

(51) Int. Cl. A61K 39/215; A61K 39/00; A61P 31/14; C07K 14/005; C12N 7/00

(52) U.S. Cl. CPC A61K 39/215; A61P 31/14; C07K 14/005; C12N 7/00; A61K 2039/53; A61K 2039/545; A61K 2039/575; C12N 2770/20022; C12N 2770/20034

(57) ABSTRACT

Provided herein are genetic vaccines and immunogenic constructs and methods of use effective to elicit immune responses against a coronavirus. The genetic vaccines and immunogenic constructs encode or comprise coronaviral spike tail peptides with one or two substitutions in the coatomer dibasic motif. Also provided are plasmids constructed to produce mRNAs for use in the genetic vaccines.

### CROSS-REFERENCE TO RELATED APPLICATIONS

This non-provisional patent application claims benefit of priority under 35 U.S.C. § 119(e) of provisional patent application U.S. Ser. No. 63/783,863, filed Apr. 4, 2025, the entirety of which is hereby incorporated by reference.

### STATEMENT REGARDING FEDERALLY SPONSORED RESEARCH

This invention was made with government support under Grant Numbers GM150187 and A1183188 awarded by the National Institutes of Health. The government has certain rights in the invention

### INCORPORATION OF SEQUENCE LISTING

The Sequence Listing XML file, entitled D8088SEQ.xml, created on Apr. 2, 2026, and with a size of 16,000 bytes, is hereby incorporated by reference in its entirety and made a part of this application.

### BACKGROUND OF THE INVENTION

### Field of the Invention

The present invention relates generally to the fields of coronaviruses and genetic vaccines. More specifically, the present invention relates to coronavirus spike protein based genetic vaccines and neutralizing antibodies that circumvent coatomer hijacking.

### Description of the Related Art

Protein secretion is a fundamental process that transports soluble and membrane proteins to intracellular and extracellular destinations in response to cellular cues. In eukaryotic cells, the secretory pathway provides the enzymes for post-translational modifications that diversify protein function (1,2). Notably, Asn- or N-glycosylation within this pathway is a key modulator of glycoprotein structure and function, which serve critical cellular functions such as protein-protein interactions (3,4). Abnormalities in secretion and N-glycosylation are linked to various disorders, including those affecting growth, immunity, and cancer (5,6).

Enveloped viruses such as coronaviruses (CoV) utilize the secretory pathway of the infected host cell for the biogenesis and N-glycosylation of the viral proteins (7). The CoV spike protein, essential for viral entry, is synthesized in the host cell ER membrane and then secreted through the ERGIC and the Golgi network, which provide enzymes for the N-glycosylation of spike luminal domain (8-10) (FIG. 1A). For instance, there are 22 N-glycosylation sites in the SARS-CoV-2 spike protein (11) (FIG. 1B). N-glycosylation of the spike protein modulates its biogenesis, receptor-binding activity, and interactions with the immune system (12-16). The trafficking of the spike between these secretory compartments is enabled by the mimicry of endogenous trafficking motifs in the spike cytosolic tail, which results in the hijacking of cytosolic carriers (17-19). For instance, a Lys-x-His-x-xCT dibasic motif (x=any amino acid, xCT=C-terminal amino acid) is reported in the cytosolic tail of the spike proteins from SARS-CoV, SARS-CoV-2, MERS-CoV, HCoV 229E and HCoV NL63, which infect humans (20-22) (FIG. 1C). This mimics the canonical Lys-x-Lys-x-xCT dibasic motif, which is recognized by the hetero-heptameric, cytosolic coatomer complex in the cytosolic tails of ER-resident membrane proteins that arrive at the cis-Golgi membrane by anterograde trafficking (23-28). Coatomer binding initiates the packaging of these “client” proteins into coatomer protein complex I (COPI)-coated vesicles for retrograde retrieval to ER to prevent the inadvertent leakage of client proteins during secretion, thereby preserving ER homeostasis (29-31). The endogenous proteins that interact with the coatomer play critical roles in essential cellular processes such as the enzyme UGT that catalyzes hormone modifications and xenobiotic detoxification, and shuttle proteins such as KDEL receptor and SURF4 that enable bidirectional secretion essential for proper cardiovascular health and immune response modulation (32-36).

Coatomer dysfunction compromises ER homeostasis, which is implicated in autoimmune diseases, cancer, and developmental disorders (36-40). From the perspective of CoVs, coatomer interactions balance the bidirectional trafficking of the spike protein from the cis-Golgi to the ERGIC for viral assembly, and from the cis-Golgi to the plasma membrane for cell-cell fusion and CoV transmission (17, 19, 41) (FIG. 1A). The spike constructs of the COVID-19 genetic vaccines include a functional dibasic motif for coatomer-interactions (42). Consequently, the coatomer is a key determinant of spike secretion and interactions with the enzymes that remodel N-glycans from immature high-mannose type to hybrid and complex type.

Prior analyses have demonstrated that the spike tail dibasic motif occupies the same binding site within the coatomer as the corresponding dibasic motif in the cytosolic tails of endogenous client proteins (25, 41, 43-45). This suggests the potential for in vivo competition for coatomer binding between the endogenous client proteins and the spike protein. In the context of minimizing the interference of spike protein-based genetic vaccines with endogenous secretion pathways, a promising approach may involve designing spike constructs that do not interact with the coatomer complex. This could represent a potentially safer alternative. However, the impact of disrupting coatomer interactions on spike N-glycosylation and subsequent interactions with host proteins remains largely unexplored although in the context of SARS-CoV-2, disrupting spike-coatomer interactions compromises spike packaging and viral fusogenicity (41).

Thus, there remain unmet needs in the art for safer vaccines directed against coronaviruses. Particularly, the prior art is deficient in anti-coronavirus spike-based genetic vaccines. The present invention fulfils this longstanding need and desire in the art.

### SUMMARY OF THE INVENTION

The present invention is directed to an immunogenic construct. The construct comprises a coronavirus spike protein with at least one substitution therein.

The present invention is further directed to an immunological composition. The composition comprises the immunogenic construct described herein and an immunologically acceptable excipient or adjuvant.

The present invention is directed further to a method for eliciting an immune response against a coronavirus in a subject in need thereof. The amount of the immunological composition described herein effective to elicit a protective antibody response is administered to the subject.

The present invention is directed further still to a plasmid. The plasmid has a nucleic acid sequence that encodes an mRNA for a mutated coronavirus spike and a promoter operably linked to the nucleic acid sequence that regulates expression of the mRNA.

The present invention is directed further still to a genetic vaccine. The genetic vaccine comprises a mRNA that encodes a mutated spike protein, wherein said spike protein, when produced in a recipient of the vaccine, elicits a protective immune response against a coronavirus.

The present invention is directed to a related genetic vaccine directed against a severe acute respiratory coronavirus 2. The genetic vaccine comprises a mRNA that encodes a mutated spike protein with a sequence comprising GVALAYT (SEQ ID NO: 2) or GVKLKYT (SEQ ID NO: 3) or a combination thereof.

The present invention is directed to a method for preventing an onset of a coronaviral infection in a subject. In this method, the subject is inoculated with a first dose of the genetic vaccine described herein effective to elicit anti-coronaviral antibody production in the subject. The present invention is directed to a related invention further comprising administering to the subject at least a second dose of the genetic vaccine.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIGS. 1A-1E show that an unmodified cytosolic tail is required for spike protein retention in early secretory compartments. FIG. 1A is an outline showing the bidirectional trafficking of the spike protein (yellow) as mediated by the coatomer complex (pink). The arrows show the sequence of anterograde trafficking from the endoplasmic reticulum (ER) to cis-Golgi (steps 1-4) and then retrograde trafficking to the ERGIC and ER (steps 5 and 6). Step 5′ shows export to the plasma membrane when the spike escapes recycling. FIG. 1B is a schematic outline of the spike protein highlighting 22 N-glycan sites and C-terminal sequence of the constructs Spike-KxH (SEQ ID NO: 1), Spike-AxA (SEQ ID NO: 2) and Spike-KxK (SEQ ID NO: 3). FIG. 1C is a sequence alignment of the C-terminal seven residues in the spike tail of SARS-CoV and SARS-CoV-2 (SEQ ID NO: 4), MERS-CoV (SEQ ID NO: 5), HCoV 229E (SEQ ID NO: 6) and HCoV NL63 (SEQ ID NO: 7) shows conservation of the coatomer-binding dibasic motif. FIG. 1D is a Western blot analysis showing cleavage of the C-terminally tagged spike-KxK due to secretion out of the early compartments. FIG. 1E shows an abundance of N-glycan species in the three full-length spike constructs. These data represent the average of three independent replicates for each construct.

FIGS. 2A-2G show N-glycan maturation at occluded sites is linked to spike recycling. FIG. 2A is structure of the full-length spike trimer highlighting an occluded N-glycan at Asn234 and an exposed N-glycan at Asn1173. FIGS. 2B-2C are MD simulations show that the access to the Asn343 N-glycan is hindered in the all-RBDs-down state. FIGS. 2D-2E show that, in contrast, the Asn343 N-glycan is exposed and accessible when the RBD is in the up-state. The N-glycans at Asn234 (buried) and Asn1173 (exposed) are shown as controls. FIG. 2F shows glycan-glycan interactions in the all-RBDs-down state occlude enzyme access to Asn616. FIG. 2G: show that the CTD1 (cyan) and CTD2 (yellow) interfere with enzyme access at Asn616 in the 1-RBD-up state.

FIGS. 3A-3N show that Spike export is a major determinant of antibody interactions. Cell culture ELISA analyses show the interactions of antibodies with the full-length, membrane-associated spike constructs. These assays were performed in triplicate and recorded over five days. The antibody VRC01 is a negative control. This shows the overall consistency between recycling spike-KxH and M-spike-KxH, and unidirectionally exported spike-AxA and M-spike-AxA constructs in antibody binding despite N-glycan differences.

FIGS. 4A-4C show the murine immunization of spike DNA vaccines elicits protective antibody response. FIG. 4A shows the murine immunization regimen for SARS-CoV-2 DNA constructs and groups. FIG. 4B shows the anti-Spike ELISA responses for each group at time zero and two weeks post the second immunization are shown. FIG. 4C shows the week 6 serum neutralization titers (ID50) for the six groups and pooled prebleed are plotted in a box and whisker format. Serum neutralizations (ID50) are shown for each group against WA-1 (blue boxes) and Asp614Gly (orange boxes) pseudo viruses. Error bars represent 95% confidence interval and neutralization ID50 were plotted on a logarithmic scale and represented by M-spike-KxH, M-spike-KxK, M-spike-AxA, Spike-KxH, Spike-KxK, and M-spike-AxA. Geometric mean titers (numerical values) are listed below box and whisker representation for each group. Statistical analyses were performed using a non-parametric Kruskal-Wallis test with Dunn's post-hoc analysis for anti-spike ELISA and Log 10(ID50) at weeks 6. Benjamini-Hochberg corrected p-values; p=>0.05 (ns); p<0.05 (*); p<0.01 (**); p<0.001 (***); p<0.0001 (****) and their corresponding significance levels are shown.

FIGS. 5A-5G show CryoEM reconstructions demonstrate the structural basis of spike-antibody interactions. FIG. 5A are single particle cryoEM maps of the full-length spike constructs show comparable 1-RBD-up and 2-RBD-up conformations despite N-glycan differences. The trans-membrane domain and the cytosolic tail are not resolved in the spike maps. FIGS. 5B-5G show N-glycan features in the spike-KxK 1-RBD-up map. The N-glycan at Asn1074 demonstrates branched features whereas the other N-glycans sites are predominantly unbranched in the cryoEM map.

FIGS. 6A-6B show interferometry data for the wild-type MHV-A59 spike tail sequence (FIG. 6A) and the sequence with alanine substitutions at the basic-acidic motif (FIG. 6B).

### DETAILED DESCRIPTION OF THE INVENTION

As used herein, the articles “a” and “an” when used in conjunction with the term “comprising” in the claims and/or the specification, may refer to “one”, but it is also consistent with the meaning of “one or more”, “at least one”, and “one or more than one”. Some embodiments of the invention may consist of or consist essentially of one or more elements, components, method steps, and/or methods of the invention. It is contemplated that any composition, component or method described herein can be implemented with respect to any other composition, component or method described herein.

As used herein, the term “or” in the claims refers to “and/or” unless explicitly indicated to refer to alternatives only or the alternatives are mutually exclusive, although the disclosure supports a definition that refers to only alternatives and “and/or”.

As used herein, the terms “comprise” and “comprising” are used in the inclusive, open sense, meaning that additional elements may be included.

As used herein, the terms “consist of” and “consisting of” are used in the exclusive, closed sense, meaning that additional elements may not be included.

As used herein, the term “about” refers to a numeric value, including, for example, whole numbers, fractions, and percentages, whether or not explicitly indicated. The term “about” generally refers to a range of numerical values (e.g., ±5-10% of the recited value) that one of ordinary skill in the art would consider equivalent to the recited value (e.g., having the same function or result). In some instances, the term “about” may include numerical values that are rounded to the nearest significant figure.

In one embodiment of the present invention, there is provided an immunogenic construct, comprising a coronavirus spike protein with at least one substitution therein.

In this embodiment, the coronavirus spike protein may comprise a substitution at one or both of the spike tail dibasic motif residues 1269Lys-Leu-His1271. In aspects of this embodiment the substitution may be 1269Lys-Leu-Lys1271 or 1269Ala-Leu-Ala. In this embodiment, the coronavirus spike protein may comprise at least one sequence selected from the group consisting of GVALAYT (SEQ ID NO: 2), GVKLKYT (SEQ ID NO: 3), PHAVAVH (SEQ ID NO: 7), PHKVKVH (SEQ ID NO: 8), VEAIAIQ (SEQ ID NO: 9), VEKIKI (SEQ ID NO: 10), FEAVAVQ (SEQ ID NO: 11), and FEKVKVQ (SEQ ID NO: 12). In addition, the coronavirus may be a severe acute respiratory coronavirus 2 (SARS-CoV-2), a Middle East respiratory syndrome coronavirus (MERS-CoV), a human coronavirus 229E (HCoV 229E), or a human coronavirus NL63 (HCoV NL63).

In another embodiment of the present invention, there is provided a immunological composition comprising the immunogenic construct as described supra and an immunologically acceptable excipient or adjuvant.

In yet another embodiment of the present invention, there is provided a method for eliciting an immune response against a coronavirus in a subject in need thereof, comprising administering to the subject an amount of the immunological composition as described supra effective to elicit a protective antibody response.

In yet another embodiment of the present invention, there is provided a plasmid comprising a nucleic acid sequence that encodes an mRNA for a mutated coronavirus spike; and a promoter operably linked to the nucleic acid sequence that regulates expression of the mRNA.

In this embodiment, the mRNA may encode at least one spike protein with a sequence selected from the group consisting of SEQ ID NOS: 2, 3, 7, 8, 9, 10, 11, and 12. Also in this embodiment the coronavirus may be a severe acute respiratory coronavirus 2 (SARS-CoV-2), a Middle East respiratory syndrome coronavirus (MERS-CoV), a human coronavirus 229E (HCoV 229E), or a human coronavirus NL63 (HCoV NL63).

In yet another embodiment of the present invention, there is provided a genetic vaccine, comprising a mRNA that encodes a mutated spike protein, wherein said spike protein, when produced in a recipient of the vaccine, elicits a protective immune response against a coronavirus.

In this embodiment, the spike protein may have a substitution at one or both of the spike tail dibasic motif residues 1269Lys-Leu-His1271. Particularly, the substitution may be 1269Lys-Leu-Lys1271 or 1269Ala-Leu-Ala. In this embodiment, the mRNA may encode at least one spike protein selected from the group consisting of GVALAYT (SEQ ID NO: 2), GVKLKYT (SEQ ID NO: 3), PHAVAVH (SEQ ID NO: 7), PHKVKVH (SEQ ID NO: 8), VEAIAIQ (SEQ ID NO: 9), VEKIKI (SEQ ID NO: 10), FEAVAVQ (SEQ ID NO: 11), and FEKVKVQ (SEQ ID NO: 12). In addition, the coronavirus may be as described supra.

In yet another embodiment of the present invention, there is provided a method for preventing an onset of a coronaviral infection in a subject, comprising inoculating the subject with a first dose of the genetic vaccine as described supra effective to elicit anti-coronaviral antibody production in the subject. In a further embodiment, the method comprises administering to the subject at least a second dose of the genetic vaccine. In both embodiments, the coronaviral infection may be a severe acute respiratory coronavirus 2 infection.

In yet another embodiment of the present invention, there is provided a genetic vaccine directed against a severe acute respiratory coronavirus 2, comprising a mRNA that encodes a mutated spike protein with a sequence comprising GVALAYT (SEQ ID NO: 2) or GVKLKYT (SEQ ID NO: 3) or a combination thereof.

Provided herein are immunogenic constructs and compositions, plasmids and genetic vaccines directed against coronaviruses. Also provided are methods of eliciting an immune response against a coronavirus or for preventing an onset of a coronaviral infection via immunization, inoculation, vaccination, or administration of the immunogenic constructs, compositions and genetic vaccines to the subject.

The present invention utilized single-site substitutions in the coatomer-binding motif in the spike cytosolic tail to arrest the full-length SARS-CoV-2 spike protein in various stages of coatomer-dependent secretion. The present invention demonstrates the pivotal role of secretory directionality in N-glycosylation and illustrates a new approach to design the next generation of spike-based genetic vaccines with direct export to the plasma membrane for immune presentation and minimal interference in the coatomer activities of the host cell.

The immunogenic constructs and genetic vaccines are based on substitutions in the coatomer-binding dibasic motif in the C-terminal seven residues in the spike tail of a severe acute respiratory coronavirus 2 (SARS-CoV-2), a Middle East respiratory syndrome coronavirus (MERS-CoV), a human coronavirus 229E (HCoV 229E), or a human coronavirus NL63 (HCoV NL63). Table 1 shows the spike tail coatomer-binding dibasic motif in the wild-type virus. Table 2 shows the dibasic substitutions in the wild-type spike tail dibasic sequence motifs.

TABLE 1
Spike tail coatomer-binding dibasic motif

| Corona- | Spike tail sequence | SEQ |
|---|---|---|
| virus | dibasic motif | ID NO: |
| SARS-CoV-2 | Gly Val Lys Leu His Tyr Thr | 1 |
| MERS-CoV | Pro His Lys Val His Val His | 4 |
| HCoV 229E | Val Glu Lys Ile His Ile Gln | 5 |
| HCoV NL63 | Phe Glu Lys Val His Val Gln | 6 |

TABLE 2
Spike tail coatomer-binding dibasic motif substitutions

| Corona- | Spike tail sequence | |
|---|---|---|
| virus | dibasic motif substitutions | SEQ ID NO: |
| SARS-CoV-2 | Gly Val Ala Leu Ala Tyr Thr | 2 |
| | Gly Val Lys Leu Lys Tyr Thr | 3 |
| MERS-CoV | Pro His Ala Val Ala Val His | 7 |
| | Pro His Lys Val Lys Val His | 8 |
| HCoV 229E | Val Glu Ala Ile Ala Ile Gln | 9 |
| | Val Glu Lys Ile Lys Ile Gln | 10 |
| HCoV NL63 | Phe Glu Ala Val Ala Val Gln | 11 |
| | Phe Glu Lys Val Lys Val Gln | 12 |

The following examples are given for the purpose of illustrating various embodiments of the invention and are not meant to limit the present invention in any fashion.

### Example 1

### Materials and Methods

### Plasmid Construction

The SARS-CoV-2 spike constructs for purification and structure determination were synthesized and cloned in a pcDNA3.1(+) vector for mammalian expression by TOPGENE Technologies (Canada). VRC7599_SARS-CoV-2_encoding full-length spike from WA-1 with two proline substitutions (46) was synthesized by Genscript and cloned into VRC8400. Full length SARS-CoV-2 D614G spike constructs with the two proline mutations and a mutated furin cleavage site used in murine immunizations were synthesized by GeneImmune and cloned into VRC8400 at Xba1 and BamH1 site. The genes for the variable heavy and light chain sequences for, CB6, CR3022, C1C-A3, S309, LY-CoV555, A23-581, LY-CoV1404, A19-46.1, A19-61.1, S2H97, BD55-5514, S2P6, and WS6 antibodies were codon optimized, synthesized and cloned into a VRC8400 (CMV/R expression vector)-based IgG1 vector as described (47). The antibody WS6 was cloned into murine Ig1 heavy and kappa vectors (48).

### Protein Expression and Purification

The SARS-CoV-2 spike constructs were transiently transfected with PEI 25k (Polyscience, USA) in Expi293F cells (Thermo Fisher Scientific, USA) maintained in Expi293F CD media (Thermo Fisher Scientific, USA). Freshly harvested Expi293F cells expressing the full-length constructs were first homogenized and then solubilized in 10% (weight by volume) n-Dodecyl-β-D-maltopyranoside (DDM; Anatrace, USA) for 1 hour at 4° C. on a Rotisserie mixer. The solubilized fraction was clarified by ultracentrifugation (rotor Type 70 Ti, speed of 40,000 rpm for 50 min), diluted by the slow addition of 2× detergent free buffer (25 mM Tris-HCl pH 7.5, 150 mM NaCl), and incubated with streptactin-XT beads (IBA Lifesciences, Germany) for 1 hour at 4° C. in a Rotisserie mixer. In contrast, the extra-cellular medium containing the ecto-domain construct was clarified by ultracentrifugation (rotor Type 70 Ti, speed 40,000 rpm for 30 min at room temperature) and incubated with the streptactin-XT resin for 1 hour at room temperature in a Rotisserie mixer. The spike protein constructs were eluted with commercial 1×BST buffer (IBA Lifesciences, Germany) and further purified on a Superose 6 16/600 size-exclusion chromatography column. The peak containing the spike trimer was used for all downstream experiments.

### Western Blots

The Western blot analysis was performed as described (41, 49). HeLa cells were maintained in Dulbecco's modified Eagle medium (Life Technologies, USA) containing glucose, and sodium pyruvate. The media was supplemented with 10% (volume/volume) FBS (Atlanta Biologicals, GA, USA), penicillin, streptomycin (Life Technologies, USA), and L-glutamine (Life Technologies, USA). The plasmids for the spike-KxH, spike-KxK, and spike-AxA constructs carrying a functional furin cleavage site were transfected and over-expressed in HeLa cells. After 24 hours, the cells were lysed, solubilized, and the clarified total cell lysate was separated by SDS-PAGE. The anti-spike mouse antibody, 1A9 (GeneTex, USA) was used to detect the intact spike protein and the cleavage product S2 by Western blotting.

### Sample Preparation for Mass Spectrometry

Four variant constructs, KxK, KxH, AxA and ECTO, were digested using 4 proteases and protease pairs, including Alpha lytic WT (Sigma, A6362. 1:100 wt/wt), Chymotrypsin (Promega, V1061. 1:10), Trypsin (Promega, V5111. 1:20)/Lys-C (Promega, VA1671. 1:20) and Trypsin (1:20)/Glu-C (Promega, V165A. 1:10). The digestion procedures were similar to those reported earlier (50) in brief: ~100 ug of each protein was performed buffer exchange with 2×400 μL of 50 mmol/L ammonium bicarbonate (ABC) in 3K Amicon Centrifugal Filter Units (Millipore, UFC5003967) at 13000 rpm/20 minutes in an Eppendorf 5430 Centrifuge to make a final volume of 40-50 uL. A final concentration of 0.1% Rapigest-SF-Surfactant (Waters, 186001861) and 20 mmol/L dithiothreitol (Sigma, 3483-12-3) were used for denaturation and reduction at 60° C. for 30 minutes. Protein alkylation was then carried out with 55 mmol/L lodoacetamide (Thermo Fisher Scientific, A39271) in 50 mmol/L ammonium biocarbonate (ABC) solution incubated at 45° C. for 45 minutes in the dark. Following alkylation, 400 uL 50 mmol/L ABC was added to mixture immediately and two buffer exchanges under the conditions as above. Approximately 2 ug proteins used with each checked for concentration and status on SDS-PAGE (4-12% NuPAGE Novex Bis-Tris Gel, Thermo Fisher Scientific, NP0321). For each protein, four samples of 10 ug were diluted in 50 μL of 50 mmol/L ABC separately and digested by sets of proteases described above. Chymotrypsin required special conditions. It was incubated at 25° C. for 2 hours. The other 3 groups were incubated at 37° C. Alpha lytic was held at 37° C. for 1 hour in 1% (ug/ul) with a second addition of 1% protease after 1 hour. Peptide digests were then desalted with MonoSpin C-18 spin columns (GL Sciences Inc., 5010-21701) and resuspended in 20 ul 10% ACN/0.1% FA (ug/ul) prior to loading into autosampler vials.

### Liquid Chromatography-Tandem Mass Spectrometry

Liquid chromatography-tandem mass spectrometry (LC-MSMS) analyses of spike protein digests were performed on an Ultimate 3000 (Thermo Fisher Scientific) in-line with an Orbitrap Fusion Lumos (Thermo Fisher Scientific) mass spectrometer. Chromatographic separation was performed using an Acclaim PepMap RSLC 75 μm×25 cm column (Thermo Fisher Scientific) at a flow rate of 300 nL/min using a gradient length of 170 min or 230 min. For the 170 min gradient, the percent of Solvent B was increased as follows (min:% Solvent B): 0:2, 25:0, 102:32, 119:80, 125:98, 135:98, 140:2, and 170:2, where solvent B is 0.1% (v/v) formic acid in acetonitrile. For the 230 min separation time, the gradient was as follows (min: % Solvent B): 0:2, 25:0, 142:32, 159:80, 165:98, 175:98, 180:2, and 230:2.

The mass spectrometer was operated in positive ion mode and using data-dependent acquisition. Settings for the ion transfer tube temperature and spray voltage were 275° C. and 1.8 kV, respectively. Instrument settings for precursor mass spectra, referred to as MS1, included a scan range of m/z 380-2000 using the standard AGC target, RF lens value of 40% and maximum injection time of 50 ms. MS1 were analyzed in the Orbitrap at a resolution of 120,000. Filters used to select precursor ions for fragmentation included charge states 2-8, minimum intensity of 50,000 and a dynamic exclusion of 15 s (precursor tolerance±10 ppm). Tandem mass spectra were acquired in “Top Speed” mode with a cycle time of 5 sec, with a standard AGC target and maximum injection time of 60 ms. Fragmentation of peptide ions was performed using stepped beam-type collision cell spectra (HCD) with collision energies of NCE 15, 25 and 35%, detected in the orbitrap at a resolution of 30,000. Those stepped HCD tandem mass spectra containing a GlcNAc oxonium ion at m/z 204.097±15 ppm among the 20 most abundant fragment ions triggered a collisionally-induced dissociation (CID) fragmentation of the same peptide precursor ion. The CID tandem mass spectra were acquired at a collision energy of 30% with an activation Q of 0.25 and activation time of 10 ms and detected in the orbitrap at a resolution of 30,000.

### MD Simulations

All simulation analyses were performed on molecular dynamics (MD) simulations conducted in previously (12, 51). These simulations are available for download at the Amaro Lab website (amarolab.ucsd.edu/covid19.php) and these shared simulation trajectories were directly utilized for analyses herein. Glycan relative exposure calculations were performed with Visual Molecular Dynamics (VMD) (52) via in-house TCL scripts. The relative exposure analysis relies on calculating the Accessible Surface Area (ASA) for each selected glycan, for each saved MD frame, in two conditions: firstly, the total surface area of each glycan (per frame) wherein protein and neighboring glycan atoms are deleted, and secondly, a true accessible surface area where protein and neighboring glycan atoms are allowed to “shield” each selected glycan (per frame). Then the relative exposure was calculated as a ratio of the shielded ASA to the ideal ASA per each glycan per MD frame. VMD's solvent accessible surface area (SASA) calculation module relies on the Shrake-Rupley algorithm (53) to calculate surface areas of user defined selections, thus this tool and its foundational algorithm were used, to calculate the surface area of each selected glycan in the ideal/total and shielded states. The Shrake-Rupley algorithm calculates surface area by rolling a probe sphere around an atom selection, and thus a probe sphere radius must be specified by the user. If the user defines a small probe radius, such as ~1.4 Å, then the resultant surface area is said to be a true “solvent accessible surface area” as this approximates the exposure of a molecule to solvent atoms, such as water atoms. Conversely, larger probe radii can be chosen to represent the degree of exposure for selected motifs to larger binding partners. For this work, a probe radius of 7.2 Å was used to approximate the N-glycan binding domain of α-1,2-mannosidase (PDB ID 1FO3) (54), the enzyme responsible for binding and cleaving oligomannose N-glycans allowing for further elaboration. Thus, calculating relative exposure of glycans with this probe radius approximates the probability of individual glycans for binding to α-Mannosidase. Glycan-glycan contacts, glycan-protein contacts, and glycan and protein root mean squared fluctuation (RMSFs) analyses were all performed with MDAnalysis tools in Python (55).

### Cell Culture ELISA Assays

HEK 293T cells (Invitrogen, CA) were thawed and incubated with growth medium at 37° C., 5% CO₂. The cells were cultured under logarithmic growth mode, and split daily for three passages until they reached logarithmic growth status. 24 hours prior to transient transfection, 100 μl of physiologically growing cells were seeded in each well of a bio-coated 96-well cell culture microplate at a density of 2×105 cells/ml. Adherent cells were lifted off using freshly prepared Trypsin-EDTA, spun down at 200×g for 5 min, and resuspended in RealFect-Medium at 70% confluency, and incubated at 37° C., 5% CO₂ for 24 hours. Prior to transfection, 40 μl of spent medium from each well was removed. DNA-TrueFect-Max complex was prepared by mixing 0.2 μg DNA in 10 μl of Opti-MEM with 0.6 μl of TrueFect-Max in 10 μl of Opti-MEM, incubated for 15 min at RT prior to transfection, then added into each well by droplet mode and gently mixed with growing cells for 5 min (or agitated at 40 rpm on a shaker). Plates were incubated at 37° C., 5% CO₂. Six to twelve hours post-transfection, 20 μl of cell growth enhancer supplemented with 20% FBA was added to each well and the plates were incubated at 37° C., 5% CO₂. Five, 96 well culture plates were transfected with each of the six spike constructs. Each individual plate was assessed in triplicate by fourteen SARS CoV-2 antibodies plus VRC01 as a negative control and blank wells at days 1 through 5.

Primary antibody solutions were prepared at 10 μg/ml with pre-warmed RealFect-Medium. Spent supernatant was removed and 90 μl of primary antibody solution at 10 μg/ml was added to each well. Plates were placed at 37° C. for 30 minutes. Goat-anti-human IgG Fc-HRP secondary antibody conjugate at 1:10,000 dilution in pre-warmed cell growth enhancer for adherent cell medium was prepared following the manufactures instruction (Jackson Immuno Research). Primary antibody solution was removed and washed thoroughly with pre-warmed RealFect-Medium. 100 μl/well of prepared secondary antibody conjugate solution was added, and the plates were incubated at room temperature for 15 minutes. Secondary antibody conjugate solution was removed, and the plates were washed twice with pre-warmed RealFect-Medium, followed by a PBS wash. 90 μl/well of TMB substrate was added, and the reaction was quenched after 10 minutes with 100 μl/well 0.5N H₂SO4 before a faint color (less than OD450=0.1000) started to develop in the negative control or medium control wells. OD450 values of cells that were transfected by the six spike constructs and incubated only with a secondary antibody were averaged and subtracted from individual wells to account for background. Data was processed further using GraphPad Prism 9.3.1.

### Murine Immunizations

Immunizations were performed in 6-to-8 week old BALB/cJ mice (Jackson Laboratories). All immunization experiments were reviewed and approved by the Animal Care and Use Committee of the VRC, NIAID, NIH and performed in compliance with US NIH regulations. Plasmid DNAs encoding SARS-CoV-2 WA-1 (aa 1-1273) with 2P mutations (M-Spike-KxH), M-Spike-KxK, M-Spike-AxA, Spike-KxH, Spike-KxK and Spike-AxA used in this study were diluted prior to immunizations in PBS pH 7.4. BALB/cJ mice were injected intramuscularly at week 0 and 4 in both hind legs with 10 μg endotoxin free plasmid DNA in 100 μl of PBS, pH 7.4, followed by electroporation at the site of injection at weeks 0 and 4. Mouse sera was collected at week 0, and 2 weeks post each DNA immunization.

### Sera Binding Analysis

Spike S2P ELISAs were performed as described (56, 57). Briefly, ELISA plates were coated with 1 μg/ml SAR-CoV-2 S2P in PBS, pH 7.4 for 16 h at 4 C. Plates were washed thrice with PBST and blocked with 5% skim milk in 1×PBST for 2 h at room temperature. Heat-inactivated week 0, 2 and 6 sera were diluted 100-fold in 5% skim milk in PBST. These were further subjected to 6-fold serial dilution for 0, 2 and 6 weeks sera. Plates were incubated with weeks 0.2 and 6 diluted sera at room temperature for 1 h. An HRP-conjugated anti-mouse secondary antibody at 1:2000 fold dilution was added and the plates incubated for 1 h at room temperature. Subsequently secondary antibody conjugate solution was removed, plates were washed thrice with PBST and 90 μl/well of TMB substrate was added, and the reaction quenched after 10 minutes with 100 μl/well 1 N H₂SO₄. The plates were read at 450 nm and data were plotted and analyzed with Graphpad Prism.

### Lentivirus-Based Pseudovirus Neutralization Assay

The pseudovirus neutralization assays were performed using published protocols (56, 58). Briefly, SARS CoV-2 pseudoviruses were obtained by co-transfecting SARS CoV-2 WA-1 spike or the D614G and KP.3.1.1 variants, a human transmembrane protease serine 2 (TMPRSS2) plasmid, lentivirus packaging plasmid pCMVd8.2, and the transducing plasmid pHR′ CMV-Luc encoding the luciferase reporter into HEK293T/17 cells (ATCC CRL-11268) using Lipofectamine 3000 transfection reagent (L3000-001, ThermoFisher Scientific, Asheville, NC). Titrated psuedoviruses were mixed with heat-inactivated sera, the mixture was incubated at 37° C. for 45 minutes and added to pre-plated 293-TMPRSS2-ACE2 cells. Cells were lysed after 72 hours and luciferase activity was measured. Percent neutralization was calculated, and ID50/ID80 titers of sera were determined using a log (agonist) vs. normalized response (variable slope) nonlinear function in Prism v9 (GraphPad 9.3.1).

### Statistical Analysis

Murine serum S2P ELISA end point titers were subjected to Kruskal-Wallis non-parametric test, followed by Dunn's test. For multiple corrections, Benjamini-Hochberg procedure with Q=0.05 to control the false discovery rate was used, ensuring no more than 5% of rejected null hypotheses are false positives on an average.

Lentivirus-based pseudovirus neutralization titers corresponding to week 2, and 6 for the six DNA constructs were initially analyzed using one-way ANOVA, however the data did not meet the assumptions of normality and homogeneity of variances, and thus not suitable for testing significant differences among groups. Data were subsequently analyzed using non-parametric Kruskal-Wallis test. Dunn's test with Benjamini-Hochberg adjustment for multiple-comparisons for pairwise comparisons was implemented when significant differences were observed. All statistical analyses were performed using R (version 4.3.3), and comparisons that yielded an adjusted p-value less than 0.05 were considered statistically significant.

Single Particle cryoEM

Purified spike protein constructs (~2-3 mg/ml, 3.5 μL) were plunge-frozen on Quantifoil R2/2 300 mesh Cu-grids, using a Vitrobot Mark IV semi-automated plunger (Thermo Fisher Scientific, USA). The grids were screened in-house on a 200 kV Talos Arctica transmission electron microscope (TEM). Final movies were collection on a 300 kV Titan Krios TEM equipped with either a K3 direct electron detector and a Bioquantum energy filter (Pacific Northwest Center for CryoEM (PNCC)), All initial processing was performed in cryoSPARC v4.4.1, including motion correction of the movies, CTF parameter estimation, particle selection using a combination of blob picking, Topaz, and template-based picking, and 2D classification (59-61). Select class averages were combined and used for ab initio model generation, followed by heterogeneous refinement. Initially, C3 symmetry was imposed during heterogeneous refinement and only selected classes that appeared complete, undamaged, and demonstrated features of a trimeric spike protein. After several iterations, the symmetry was relaxed to C1 during heterogeneous refinement. This yielded multiple different conformational classes of the spike protein. Each of these conformational states was used for homogeneous and non-uniform refinement. After cleaning up these data, all the high-resolution reconstructions and the spike particles were exported to RELION 3.1.2, for 3D classification for a final separation of the conformational states by 3D classification (62). The particles for the various RBD up-down states were imported back into cryoSPARC v4.4.1, and used in homogeneous and non-uniform refinement. Finally, refinement of local and global CTF parameters was performed.

The spike coordinates from previous structures (PDB ID 7KRR and 7KRQ) (63) were fitted in the spike cryoEM maps in Chimera. A combination of Chimera, COOT, and phenix.realspace.refine were used for iterative model building (63-65).

### Superposition of Fab-RBD Structures

The structural basis of interactions between the spike constructs and the Fab fragments by superimposition was analyzed in PyMol using the following PDB IDs for the Fabs-7SN2 (C1C-A3), 6W41 (CR3022), 7KMG (LY-CoV555), 7LRS (A23-58.1), 7M7W (S2H97), 7MMO (LY-CoV1404), 7RNJ (S2P6), 7TBF (A19-61.1), 7TC9 (A19-46.1), 7TCQ (WS6), 7XCK (S309), and 7Y0W (BD55-5514).

### Example 2

### Results

### An Unmodified Cytosolic Tail is Required for Spike Protein Retention in Early Secretory Compartment and Remodeling of the N-Glycan Landscape

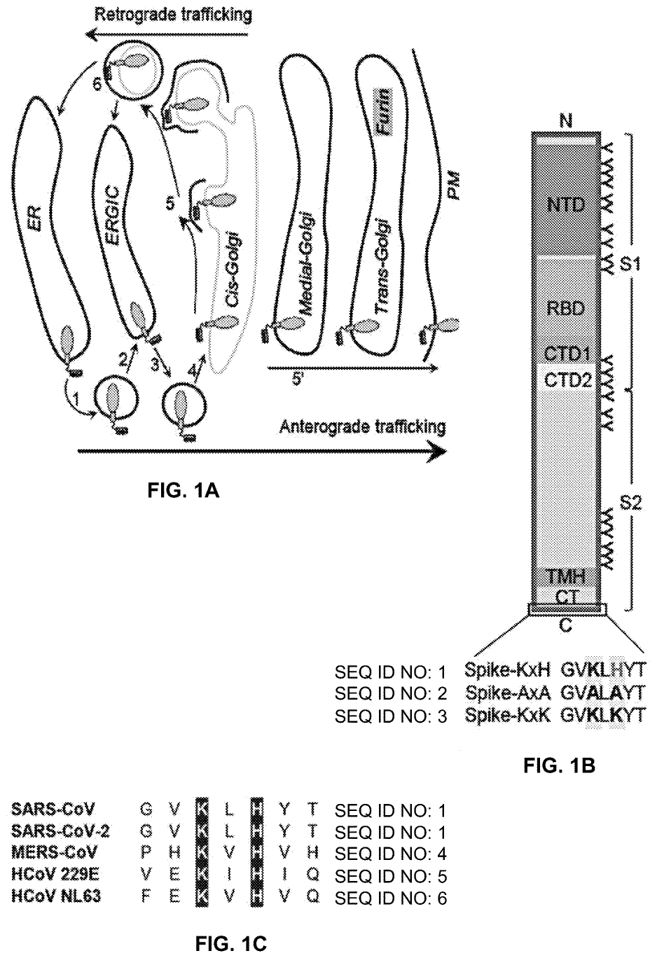

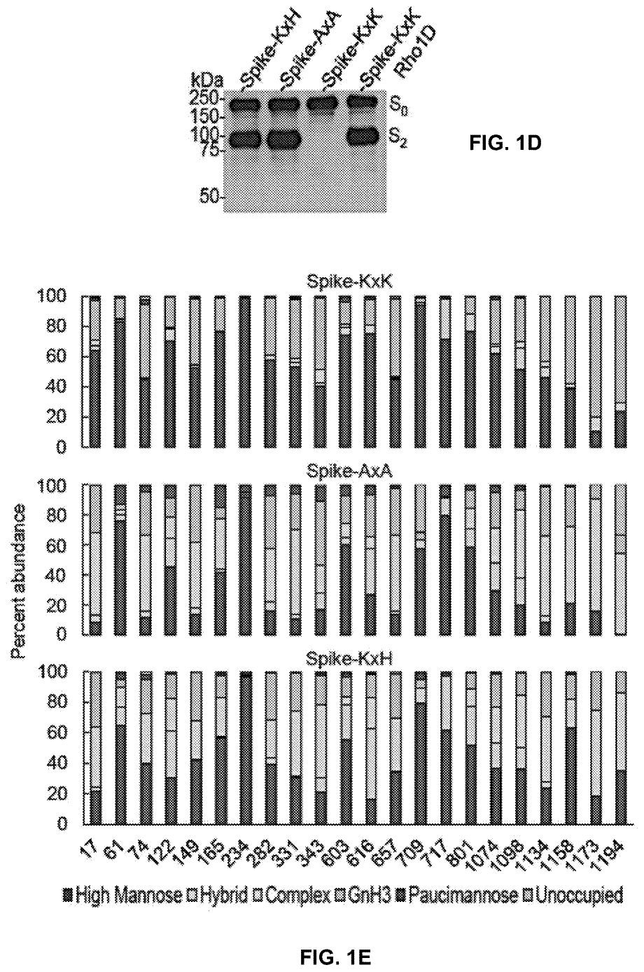

The aim was to arrest the spike protein, (i) in early secretory compartments prior to export, (ii) during recycling in the early secretory compartments and then export to the plasma membrane, and (iii) upon a single transit to the plasma membrane without recycling. The tail was mutated at 1269Lys-Leu-His1271 dibasic motif in the wild-type spike construct (“spike-KxH”) to 1269Lys-Leu-Lys1271 sequence, which retains the spike in early secretory compartments (henceforth called the spike-KxK construct; FIG. 1B). To generate the construct that undergoes a single transit to the plasma membrane without coatomer-dependent recycling, the dibasic motif was substituted to 1269Aa-Leu-Ala1271 (henceforth called the spike-AxA construct; FIG. 1B).

Next, potential sites were evaluated for spike tagging for affinity purification. Prior studies with dibasic motif peptides have shown that a free C-terminus in client proteins such as the spike protein is essential for coatomer interactions (25, 41). Hence, it was hypothesized that the widely employed C-terminal affinity tagging strategy would interfere with spike secretion (62, 66). To test this, a full-length spike-KxK construct was generated with a C-terminal Rho1 D purification tag and conducted a Western blot analysis to evaluate its cleavage by the protease furin, which is encountered in the trans-Golgi if the spike escapes the early secretory compartments (FIG. 1A). Cleavage of this tagged spike-KxK construct was observed whereas the untagged spike-KxK construct shows a single uncleaved band and thus is inferred to be correctly retained in early secretory compartments (FIG. 1D). Hence, it was concluded that the C-terminal tagging strategy is incompatible with the objectives on spike trafficking and that the tag for affinity purification will have to be inserted elsewhere in the spike.

Having established the essential role of an untagged cytosolic tail in coatomer-dependent spike secretion, we adopted our recently developed internal-tagging strategy to generate full-length, 1273 residue, membrane-associated spike-KxH, spike-KxK, and spike-AxA constructs that have 22 N-glycan sites (FIG. 1B). Briefly, a twin-strep tag was inserted downstream of the N-terminal signal sequence in the full-length spike gene encoding the ecto-domain, trans-membrane domain, and the cytosolic tail. After transient transfection in Expi293F cells and purification in the detergent, n-dodecyl-β-D-maltopyranoside (DDM), the spike-KxH, spike-KxK, and spike-AxA proteins were subjected to N-glycan analysis by electro-spray ionization mass spectrometry. Each sample was digested in seven different ways to generate a glycopeptide abundance distribution spectrum for each of the N-glycosylated ‘sequons’. Here, the mass of each peak corresponds to the glycan mass and glycopeptide abundance is related to relative abundance in the electro-spray ionization mass spectrum.

The analysis of the spike-KxK construct shows an abundance of immature, high-mannose N-glycans and hybrid glycans (FIG. 1E). This observation aligns with its localization within the early secretory compartments, prior to exposure to enzymes responsible for trimming and modifying N-glycans (49). Moreover, this N-glycan profile is consistent with the immature N-glycans observed in the early endomembrane retained subset of spike molecules of the ChAdOx1 nCoV-19/AZD1222 genetic vaccine developed by AstraZeneca (67). Consequently, the spike-KxK construct can be characterized as the endomembrane-retained glycoform of the spike-based genetic vaccine. In contrast, substantial N-glycan processing is seen in the spike-KxH and spike-AxA constructs, which is broadly consistent with the exported spike molecules of the ChAdOx1 nCoV-19/AZD1222 genetic vaccine (67). Given that both spike-KxH and spike-AxA ultimately localize to the plasma membrane, we hypothesized that they would undergo similar secretory processing, resulting in comparable levels of mature N-glycans. However, we found several differences between the spike-KxH and spike-AxA constructs. For instance, the sequons Asn17, Asn74, Asn149, Asn165, Asn282, Asn331, Asn657, Asn1158, and Asn1173 contain 1.4-to-2.7-fold more complex N-glycan in spike-AxA than in the spike-KxH construct (FIG. 1E). These sites are well-exposed in the trimeric spike with unobstructed accessibility to the solvent, and likely to the N-glycan modifying enzymes. Hence, we concluded that secretory routing is a direct determinant of N-glycan maturation at sites that are accessible on the spike surface.

### N-Glycan Maturation at Occluded Sites is Linked to Spike Recycling

It was next hypothesized that since the spike-AxA construct localizes predominantly in the plasma membrane because it does not recycle, all its sequons must contain a larger, or at least comparable amount of complex N-glycans relative to spike-KxH. However, contrary to expectation, the Asn343 sequon, which gates RBD conformational changes (15) showed a ~2.6-fold excess of complex N-glycans in spike-KxH over the corresponding spike-AxA Asn343 sequon (FIG. 1E). We hypothesized that the RBD impacts the Asn343 N-glycan accessibility, thus limiting its interactions with glycan processing enzymes. To test this hypothesis, molecular dynamics (MD) simulations were used and determined the in silico accessibility of these N-glycans to the enzyme, α-1,2-mannosidase, which catalyzes the early hydrolytic trimming of terminal mannose groups from immature high-mannose precursors in glycoproteins (54, 68, 69). α-1,2-mannosidase is a transmembrane protein, with its C-terminal catalytic domain oriented towards the secretory lumen, which also houses the N-glycosylated spike ecto-domain. Proper access to the catalytic site of α-1,2-mannosidase is crucial for mannose trimming, thereby initiating N-glycan maturation.

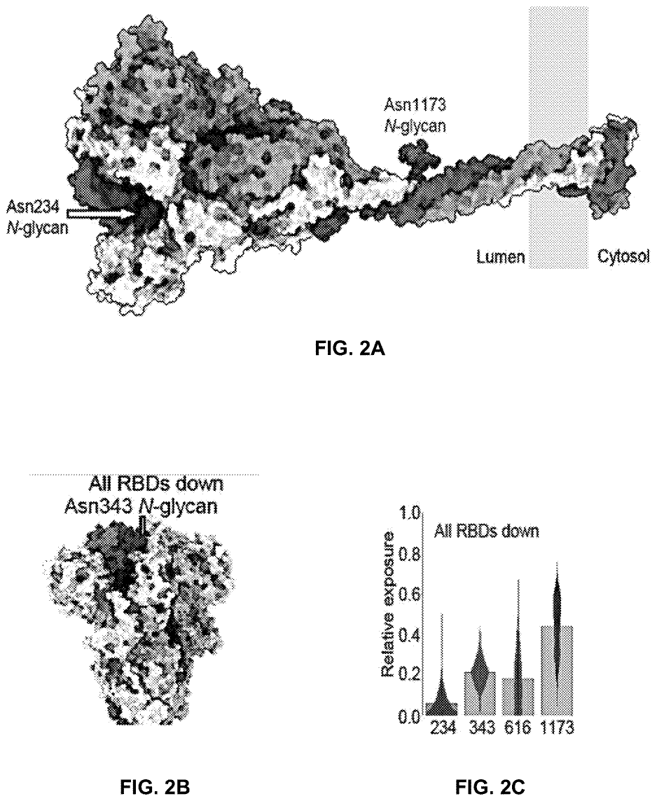

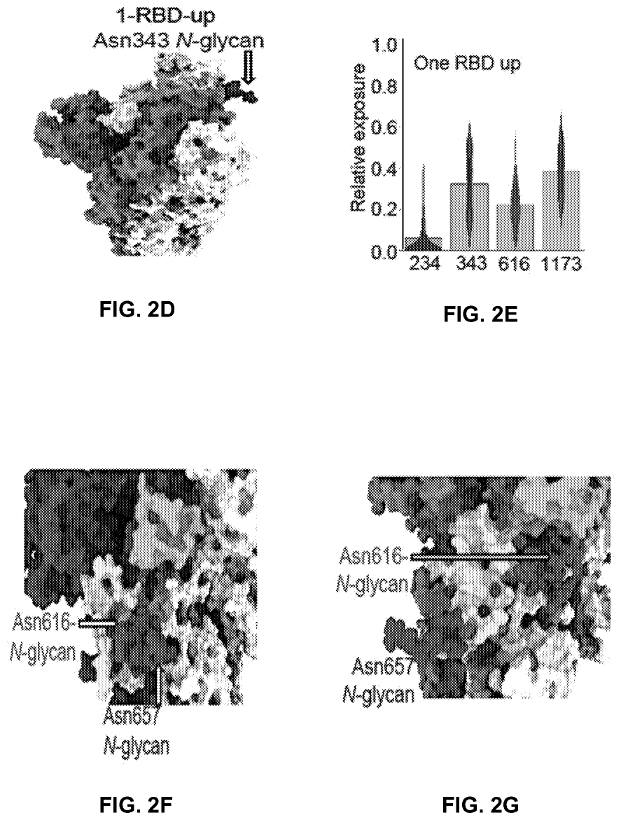

To assess the accessibility of the Asn343 N-glycan, a relative accessible surface area of this N-glycan was calculated for every frame in previously reported MD simulations (12). Accessible surface area (ASA) was calculated according to the Shrake-Rupley algorithm, as implemented in Visual Molecular Dynamics (VMD), where a probe sphere of user defined radius is “rolled” along a molecular surface to determine area (53). A probe sphere of radius 7.2 Å was defined to approximate the active site binding cavity of α-1,2-mannosidase (PDB ID 1FO3 (54). Relative accessibility per frame was calculated as the ratio of the accessible surface area of Asn343 N-glycan when considering neighboring spike glycoprotein atoms, to the ASA of the Asn343 glycan when all other spike glycoprotein atoms are ignored, i.e. an “ideal” surface area if the Asn343 N-glycan in that conformation was fully solvated. Calculating relative accessibility according to this method can approximate whether an N-glycan in a given conformation is likely to be exposed enough to bind to α-1,2-mannosidase as ratios of ~1 indicate the Asn343 N-glycan in an exposed conformation likely capable of binding alpha-1,2-mannosidase, while ratios close to ~0 indicate a highly shielded N-glycan conformation likely not capable of binding α-1,2-mannosidase. The N-glycans at Asn234 and Asn1173 were included as controls because they provide limiting cases of a well-shielded and a well-exposed glycan, respectively (FIG. 2A). The analysis from prior MD simulations (12) showed that in the all-RBDs-down state, the Asn343 N-glycan is sandwiched between two adjacent RBDs, which limits its surface accessibility (FIGS. 2B-2C). In contrast, the transition of the RBD to the up-state exposes the Asn343 N-glycan on the surface, thus playing a pivotal role in modulating its maturation (FIGS. 2D-2E).

This analysis has two major implications for the spike protein structure. First, it was inferred that the spike RBDs are predominantly in the down-state during secretory recycling and export because the Asn343 sequon shows a substantial proportion of immature N-glycans. Second, steric barriers to glycan-modifying enzymes result in inefficient N-glycan processing during unidirectional export and that repeated encounters with these enzymes during recycling enhance N-glycan processing. If this hypothesis holds true, then recycling may represent a generalizable strategy for N-glycan maturation at sterically occluded sites in the spike protein. To test this hypothesis, the mass spectrometry data was analyzed and identified the N-glycan at Asn616 as a second site that demonstrates enrichment of more mature hybrid and complex glycans in spike-KxH relative to spike-AxA (FIG. 1E). Our MD simulations approach revealed multiple structural modulators of Asn616 N-glycan accessibility. First, it was observed that in the all-down state of the RBDs, the Asn616 N-glycan is buried in a valley surrounded by the C-terminal domain 2 (CTD2), its adjacent furin cleavage site, and the N-glycan at Asn657 (FIG. 2F). Second, the MD analysis showed extensive glycan-glycan interactions between the Asn616 and Asn657 sites (FIG. 2G). This interaction shields the Asn616 N-glycan, thus restricting its access to the N-glycan modifying enzymes. Third, and finally, it was observed the insertion of the CTD2 domain between the Asn657 and Asn616 sites (FIG. 2G). This wedge further restricts the access of the Asn616 N-glycan to the N-glycan modifying enzymes. Hence, our mass spectrometry and MD simulations analyses indicate that secretory recycling enhances N-glycan maturation by providing multiple encounters with the N-glycan modifying enzymes. Collectively, this investigation has revealed fundamental functions of the secretory route taken by the spike in modulating N-glycan maturation.

### Spike Export is a Major Determinant of Antibody Interactions

Site-specific deletions have shown that N-glycans modulate spike RBD conformations (12, 15, 70, 71). However, whether secretory remodeling of these N-glycans impacts spike conformations is not well understood. To investigate whether N-glycan remodeling due to the disruption of the native, coatomer-dependent recycling affects spike conformations, cell surface based ELISAs were conducted on spike constructs exported to the cell surface using a panel of monoclonal antibodies that target distinct epitopes in the S1 and S2 subunits. Untagged spike-KxH and spike-AxA constructs were expressed, and the corresponding constructs of the Moderna SARS-CoV-2 vaccine backbone, M-spike-KxH, and M-spike-AxA. Our spike constructs include two substitutions that are not in the Moderna constructs: Asp614Gly substitution for enhanced spike stability, and Arg-Arg-Ala-Arg684→Gly-Ser-Ala-Ser684 substitution to disrupt the furin cleavage site (46, 72). All constructs have an Asp-Lys987→Pro-Pro987 substitution for spike stabilization (46).

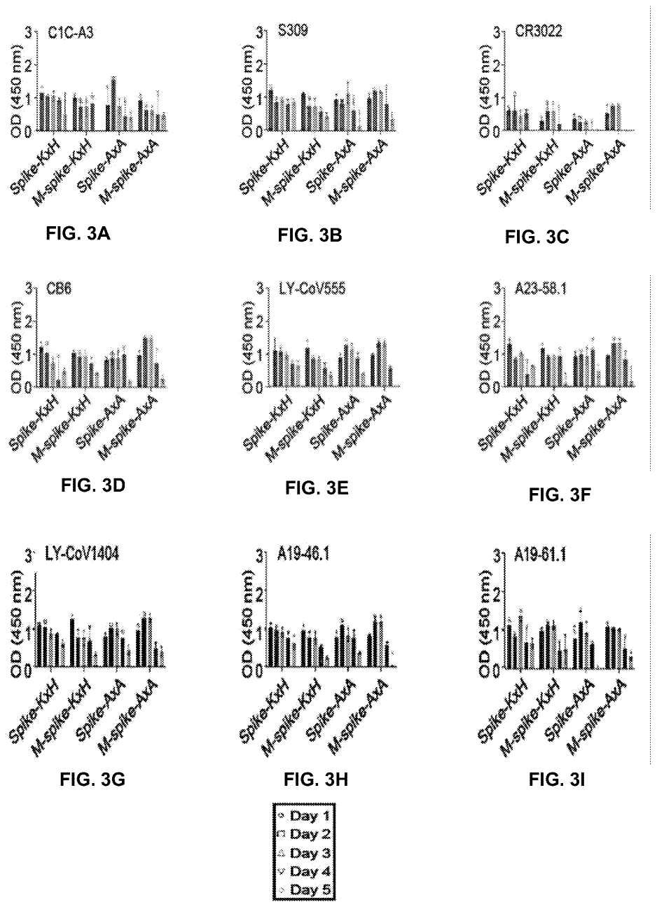

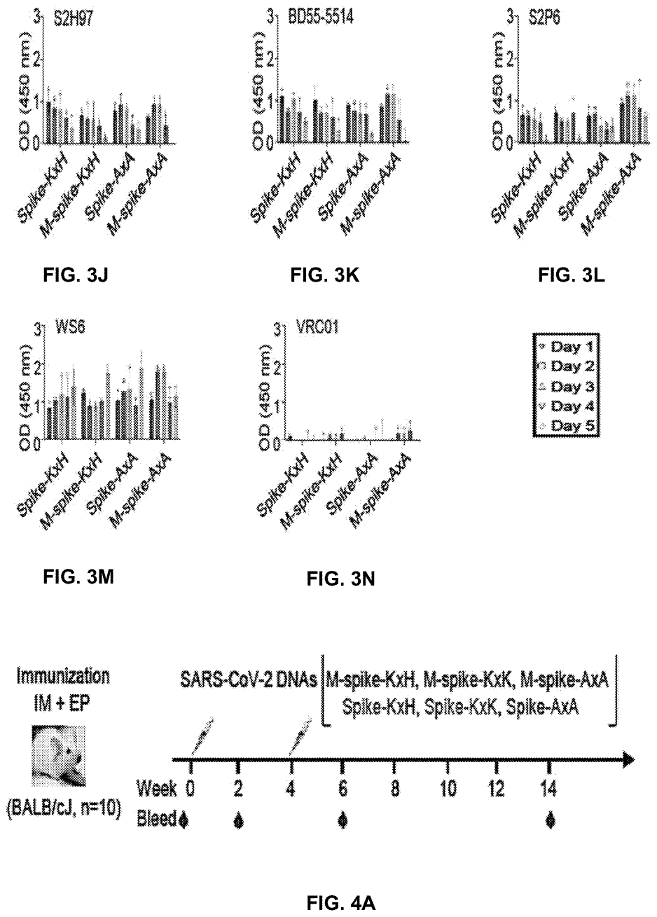

It was asked if N-glycan changes directly impact antibody binding. For this purpose, the antibodies C1C-A3 (73) and S309 (74) were tested, which interact directly with the Asn343 N-glycan. Comparable antibody binding was observed to all four spike constructs exported to the plasma membrane (FIGS. 3A-3N). Hence, it was concluded that N-glycan changes do not directly impact antibody binding.

Next, it was asked if changes in N-glycans impact the RBD up-down conformations. To address this question, the antibody CR3022, whose cryptic epitope is exposed when two of the three RBDs are in the up-state, was used (75) (FIGS. 3A-3N). Comparable antibody binding to all the four spike constructs was observed. Hence, these findings indicate that alterations in N-glycans do not substantially impact the distribution of up- and down-state RBD populations to a degree that would affect epitope presentation to the antibody CR3022. Additionally, the overall binding affinity of CR3022 to the various spike protein constructs was notably lower compared to the antibodies C1C-A3 and S309. These findings indicate that, irrespective of N-glycan variations, the cryptic epitope recognized by CR3022 remains consistently rare in its exposure across all spike protein constructs analyzed.

Having established that the spike-antibody interactions are independent of direct interference from N-glycan changes, it was determined if N-glycan changes indirectly impact antibody binding such as through conformational effects. Hence, the local conformations and accessibility of the spike S1 and S2 subunits in the vicinity of N-glycan sites were investigated, employing a panel of antibodies with distinct footprints and approach angles on the RBD. CB6 is a class I antibody that directly binds and occludes the ACE2 receptor-binding site when the RBD is in the up-state (76) ELISA analysis revealed that the M-spike-AxA construct exhibited the highest level of CB6 interaction, with peak binding observed on days 2 and 3 post-transfection (FIGS. 3A-3N). In contrast, CB6 interaction with the spike-AxA construct remained relatively consistent from days 1 to 4. Overall, both spike-AxA and M-spike-AxA constructs demonstrated comparable or enhanced CB6 binding compared to their respective KxH constructs. This trend of comparable or improved antibody interaction with spike-AxA and M-spike-AxA constructs extends to class II antibodies LY-CoV555 and A23-58.1, which target the RBD in both up- and down-state conformation (58, 77), and to S309, LY-CoV1404, and A19-46.1, which are class III antibodies whose footprint is on the RBD face opposite the class I and II footprint (78, 79). Collectively, these results suggest that epitope accessibility on the spike RBD is largely unaffected by N-glycan variations.

Next, the dependence of cryptic epitope accessibility on N-glycan differences was tested. For this purpose, A19-61.1, a class IV antibody that binds a cryptic, conformation-specific RBD epitope (79), and S2H97 and BD55-5514, which are class V antibodies that binds a cryptic epitope distinct from the class IV epitope were used (80, 81). Binding assays revealed comparable interactions of these antibodies with all four spike constructs (FIGS. 3A-3N). These findings align with previous observations for the CR3022 antibody, which also binds a cryptic epitope. Collectively, these results suggest that spike conformations and epitope accessibility are unaffected by N-glycan variations.

The assays described above do not provide direct insights into the conformational state or accessibility of the S2 subunit, as the antibodies used exclusively target the S1 subunit. To investigate these aspects, we conducted ELISA analyses of the four constructs using two S2 subunit-specific antibodies, S2P6 and WS6 (48, 82). The results revealed comparable antibody binding across all four constructs, despite furin cleavage being inactivated in only two constructs, spike-KxH and spike-AxA. In contrast, the other two constructs, M-spike-KxH and M-spike-AxA, retain susceptibility to S1/S2 cleavage. Interestingly, WS6 exhibited higher binding than S2P6, despite their partially overlapping epitopes. This discrepancy may result from differences in the binding angles of the two antibodies to the spike S2 subunit. Collectively, these cellular ELISA analyses demonstrate that secretory spike export rather than the exact N-glycan composition is a major determinant of antibody interactions, and that disruption of native, coatomer-dependent recycling does not compromise the epitope accessibility in the spike protein.

### Murine Immunization of Spike DNA Vaccines Elicits Protective Antibody Response

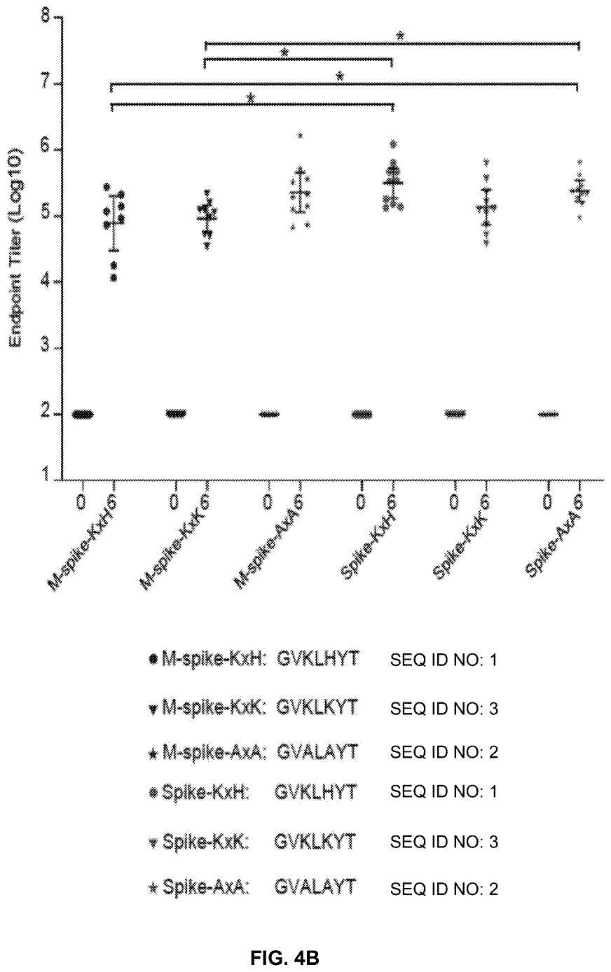

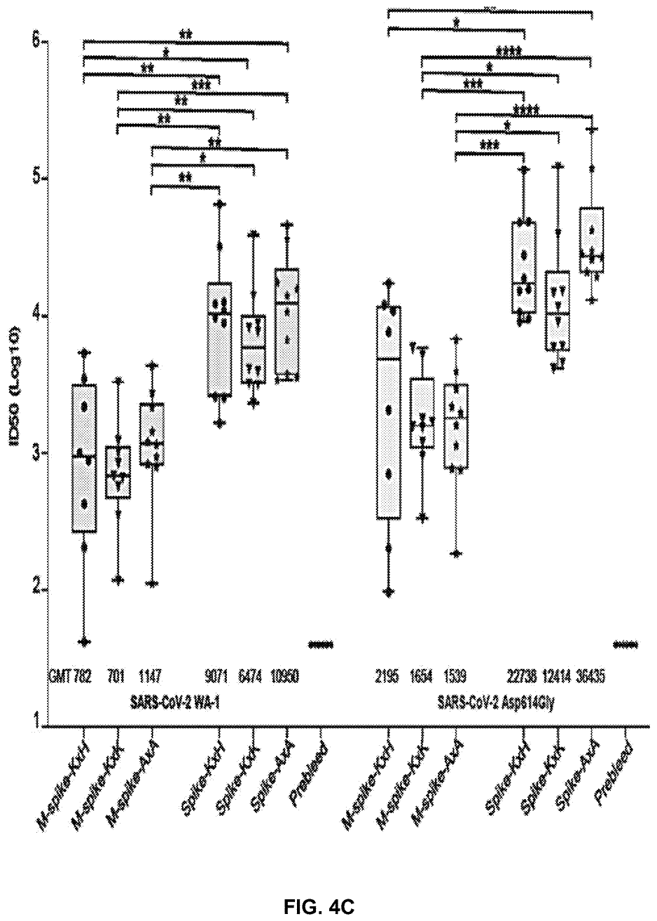

Efficient secretion of the spike protein to the cell surface is essential for its optimal presentation to the immune system (42). However, the role of native recycling prior to surface export in preserving the stability of the displayed spike protein remains unclear. Additionally, the consequences of disrupting this recycling pathway on the immunogenicity of the spike protein have yet to be determined. To explore the effect of altering the recycling capability and its impact on the immunogenicity of spike protein, a panel of all six DNA immunogens that included spike-KxH, spike-KxK, spike-AxA, M-spike-KxH, M-spike-KxK, and M-spike-AxA (FIG. 4A) was assembled.

6- to 8-week-old BALB/cJ mice were immunized with plasmid DNA encoding the six spike constructs. Intramuscular immunizations followed by electroporation were performed at week 0 and 4 (FIG. 4A). Immune responses to WA-1 spike were monitored two weeks post each immunization at week 2 and 6 by ELISA (FIG. 4B). Overall, spike ELISA titres for all six groups increased from week 2 to week 6. ELISA titers for all groups post first immunization were mostly comparable except for spike-KxH group which elicited significantly higher response than spike-KxK (P=0.042) and M-spike-AxA (P=0.042) responses. ELISA response post second immunization was significantly higher in groups immunized with spike-KxH and spike-AxA compared to groups immunized with M-spike-KxK (FIG. 4B, P=0.02, and P=0.025) and M-spike-KxH (FIG. 4B, P=0.025, and P=0.046).

Immune response at week 2 and week 6 also were monitored for all six groups by testing serum neutralization of SARS-CoV-2 WA-1, Asp614Gly, and KP.3.1 pseudo-viruses. At the earlier time point, geometric mean titers against SARS-CoV-2 WA-1 and Asp614Gly pseudo-viruses ranged in 50% inhibitory dose (ID50) from 47 to 283 and 48 to 414, respectively. The groups immunized with spike-KxH (GVKLHYT, SEQ ID NO: 1), spike-KxK (GVKLKYT, SEQ ID NO: 3) and spike-AxA (GVALAYT, SEQ ID NO: 2), elicited a higher response as compared to the M-spike-KxH (GVKLHYT, SEQ ID NO: 1), M-spike-KxK (GVKLKYT, SEQ ID NO: 3), M-spike-AxA (GVALAYT, SEQ ID NO: 2) groups. Groups that were immunized with spike-KxK elicited a lower response with geometric mean titers of 195 and 302 ID50 and 84 and 127 ID80, suggesting that possible retention of the spike protein in early secretory compartment prior to export limited the immune response.

Overall, neutralization titres for all six groups increased from week 2 to week 6. Geometric mean titers at week 6 against SARS-CoV-2 WA-1 and Asp614Gly variant ranged from 701 to 10950 ID50 and 1539 to 36435 ID50, respectively. The groups immunized with spike-KxH, spike-KxK, and spike-AxA, which carry the Asp614Gly substitution and are resistant to S1/S2 proteolysis, elicited higher immune response as compared to groups immunized with M-spike-KxH, M-spike-KxK, M-spike-AxA (FIG. 4C). The group immunized with spike-AxA elicited the highest immune response with a geometric mean titer of 10950 and 36435 against SARS-CoV-2 WA-1 and Asp614Gly, respectively, suggesting that this S1/S2 proteolysis resistant construct where the dibasic motif has been disrupted by an Ala1269/Ala1271 substitution might be effective in eliciting strong anti-spike directed immune response in mice.

Significant differences were observed in the immune response against SARS-CoV-2 WA-1 between groups immunized with spike-AxA and M-spike-KxH (P=0.0014), M-spike-KxK (P=0.0003), and M-spike-AxA (P=0.0014), respectively (FIG. 4C). A significant difference was observed in the immune response against the Asp614Gly variant between groups immunized with spike-AxA and M-spike-KxH (P=0.0018), M-spike-KxK (P=0.0001), and M-spike-AxA (P=0.0001), respectively (FIG. 4C). We observed a similar trend in the groups immunized with spike-KxK and spike-KxH immune responses when compared to the groups immunized with M-spike-KxH, M-spike-KxK, M-spike-AxA against both SARS-CoV-2 WA-1 and Asp614Gly pseudo-viruses (FIG. 4C). These trends in neutralization titers suggest that altering the recycling ability along with using the S1/S2 proteolysis resistant, Asp614Gly variant impacts the immunogenicity of the spike protein in mice. Serum neutralization also was tested against one of the more recent KP.3.1 variants, and as with the wild-type M-spike-KxH found that none of the groups neutralized this variant.

### CryoEM Reconstructions Demonstrate Structural Basis of Spike-Antibody Interactions

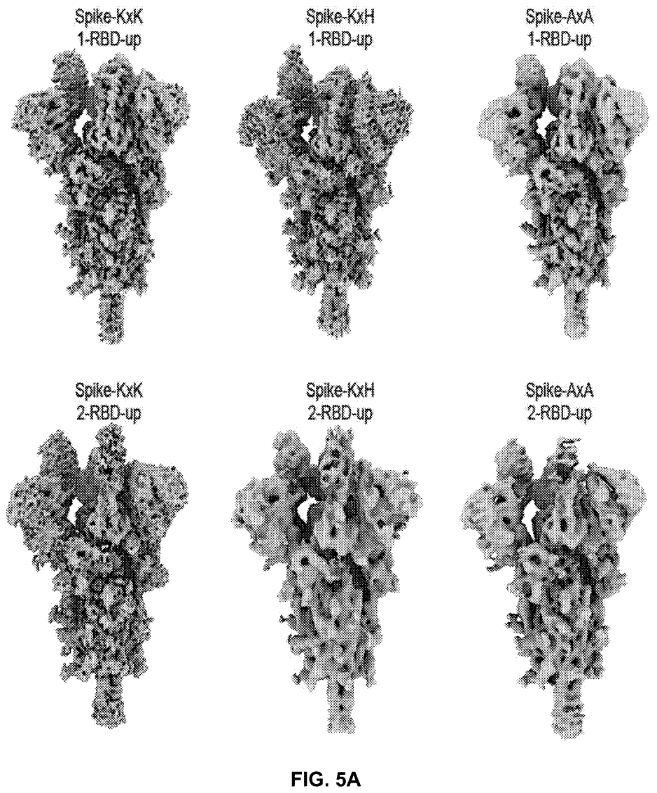

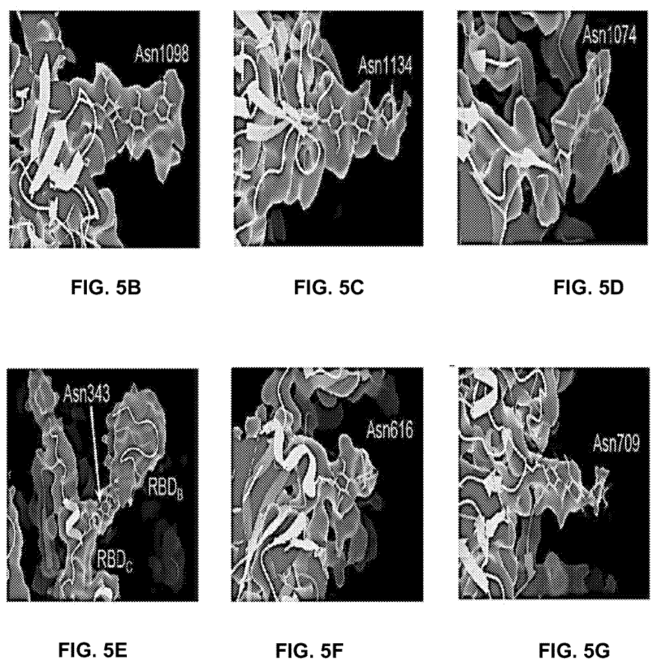

Having shown the role of the secretory route in spike-antibody interactions, we asked if N-glycan modifications due to distinct secretion of the various constructs alters the spike structure and conformations. Hence, single particle cryoEM reconstructions of the spike-KxH, spike-KxK, and spike-AxA constructs were performed, which yielded two major conformations with either one or two RBDs in the up-state (FIG. 5A). The 1-RBD-up conformation structures were determined to resolutions of 2.9 Å, 2.9 Å, and 3.3 Å for spike-KxK, spike-KxH, and spike-AxA, respectively, whereas the 2-RBD-up conformation structures were determined to resolutions of 3.0 Å, 3.8 Å, and 4.2 Å for spike-KxK, spike-WT, and spike-AxA constructs, respectively (FIG. 5A). These reconstructions showed similar structural features, as inferred from Ca RMSD of 0.2-1.6 Å for the 1-RBD-up state and 0.6-1.0 Å for the 2-RBD-up state. A consistent 4-fold abundance of the spike-KxK, spike-KxH, and spike-AxA particles in the 1-RBD-up conformation over the particles in the 2-RBD-up conformations was observed. This smaller proportion of the spike 2-RBD-up conformation is consistent with the above-reported weaker response in CR3022 binding, which requires a 2-RBD-up conformation. The 4-fold abundance of the 1-RBD-up state means that there is a relative abundance of RBDs in the down-state in all the spike constructs. Specifically, the cryoEM data suggest that the RBDs in the down-state constitute 60% of the population whereas the RBDs in the up-state constitute 40% of the population. This is consistent with the inferences about spike RBD conformations drawn above from the abundance of immature N-glycans at Asn343. In these reconstructions, large portions of the CTD2 domain were poorly ordered, suggesting conformational flexibility. This is consistent with the above-reported MD simulations showing their conformational rearrangement, including near the furin cleavage site within the CTD2 domain. Further analysis showed that the internal twin-strep affinity purification tag was disordered in all the spike constructs. The C-terminal portion of the spike such as the stem helices, the transmembrane domain, and the C-terminal cytosolic tail are disordered in all the cryoEM maps. Overall, these cryoEM reconstructions are suggestive of consistent features and conformations in the various spike constructs despite N-glycan differences.

Next, the structural basis of spike interactions with the antibodies tested in the previous section was analyzed. The superposition of previously published structures showed that the upright RBD in the 1-RBD-up state is compatible with interactions with the Fab fragments of CB6, LY-CoV555, A23-58.1, LY-CoV1404, A19-46.1, A19-61.1, and BD55-5514. This is consistent with the spike-antibody binding assays in the previous section. The Fab S309 orientation is competent for binding the 1-RBD-up state apart from a few minor clashes. In contrast, substantial clashes between an adjacent down state RBD and the Fab C1C-A3 was observed, but these clashes are absent in the 2-RBD-up state wherein the previously clashing down state RBD underwent a conformational change to the up-state. In contrast, the superimposed Fabs CR3022 and S2H97 demonstrate clashes with the spike protein in both the 1-RBD-up and 2-RBD-up states. The epitopes of the antibodies WS6 and S2P6 in the S2 stem helices are disordered and not visualized in our cryoEM maps. The overall dichotomy between the binding assays for the antibodies CR3022 and S2H97, and cryoEM reconstructions suggests the presence of additional, antibody-binding competent states of the spike constructs other than the reconstructions reported here. This inference is supported by conformational changes in the N-terminal domains, RBDs, and the S2 stem-helix observed in a 3D variability analysis of the 1-RBD-up spike protein. These are strongly indicative of such additional states for antibody interactions. Further support is provided by the weaker map features around the second up-state RBD in the spike 2-RBD-up states, which suggests additional conformational states.

Finally, a structural analysis of the spike N-glycans was performed. Overall, more extensive coverage of the S2 subunit N-glycans was observed than in the S1 subunit. This is likely indicative of the more extensive conformational stability of the S2 subunit and of the dynamics of the S1 subunit in the NTDs and RBDs. The S2 N-glycans proximal to the base at Asn1098 and Asn1134 form well-defined and extended chains (FIGS. 5B-5C) whereas N-glycan at Asn1074 demonstrates branching rather than a linear structure in the spike-KxK reconstruction (FIG. 5D). In contrast to the S2 N-glycans, we observed fewer features in the S1 N-glycans. In the S1 subunit, the Asn343 N-glycan is intercalated between two adjacent, down-state RBDs (FIG. 5E). This is consistent with the MD simulations data showing limited accessibility of the Asn343 N-glycan in the down-state. The N-glycans in the more C-terminal regions of the S1 subunit were better defined in the cryoEM maps, such as at Asn616, which is adjacent to the fusion loop proximal region and the furin-cleavage site (FIG. 5F). Another example is the N-glycan at Asn709, whose well-defined extended chain is proximal to the extended N-glycan chains at Asn1098 and Asn1134 (FIG. 5G). Collectively, these cryoEM analyses defined site-specific structural features in the N-glycan sites, consistent with MD simulations and antibody interaction data above.

### Example 3

### Novel Basic-Acidic Motif in the Coronavirus Spike Tail for Coatomer Binding

The spike protein tail of coronaviruses such as SARS-CoV-2 displays a Lys-x-His motif, which mimics the canonical dibasic motif for coatomer binding in endogenous cellular clients of the coatomer. However, it was discovered that there are coronaviruses whose spike tail lacks this dibasic motif but displays a basic-acidic motif (83). Examples include the murine hepatitis virus A59 (MHV A59), human coronaviruses HKU1 (HCoV-HKU1) and OC43 (HCoV-OC43), and the bovine coronavirus Mebus (BCoV-Mebus). Table 3 shows the C-terminal residues of these spike proteins, with the basic-acidic residue underlined. It was discovered that this basic-acidic sequence is a functional coatomer binding motif.

TABLE 3
Spike tail coatomer-binding basic-acidic motif

| Corona- | Spike tail sequence | |
|---|---|---|
| virus | basic-acidic motif | SEQ ID NO: |
| MHV-A59 | Ser Ser His Glu Asp | 13 |
| HCoV-HKU1 | Ala Ser His Asp Asp | 14 |
| HCoV-OC43 | Thr Ser His Asp Asp | 15 |
| BcoV-Mebus | Thr Ser His Asp Asp | 15 |

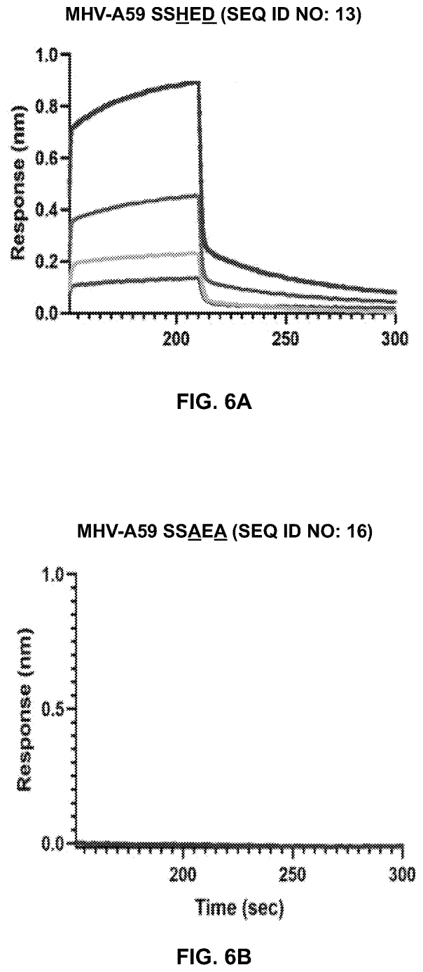

FIGS. 6A-6B show biolayer interferometry data wherein peptides encoding the MHV-A59 spike tail sequence were immobilized on a biosensor and then exposed to the αWD40 domain of the coatomer. FIG. 6A shows binding of the coatomer domain to the wild-type peptide sequence whereas FIG. 6B shows that substitution of the basic-acidic sequence with alanine residues (Ser Ser Ala Glu Ala; SEQ ID NO: 16) disrupts this interaction.

Therefore, we have discovered a novel coatomer binding motif in the spike protein of coronaviruses. Collectively, the data on SARS-CoV-2 spike dibasic motif and MHV basic-acidic motif show that the Hasan lab has established a unique and universal approach to disrupt coronavirus spike-coatomer interactions. This may be of broad applicability in the design of spike-based anti-coronavirus vaccines.

### DISCUSSION

The present invention demonstrates the impact of secretory recycling and export of the SARS-CoV-2 spike protein on its N-glycosylation, structural properties, interactions with antibodies, and overall immunogenicity. A structure-guided internal tagging approach was employed to generate spike constructs suitable for recombinant production in Expi293F cells and purification. Modifications to the coatomer-binding motif in these constructs allowed for either retention within endomembrane compartments or direct export to the plasma membrane, circumventing secretory recycling. This approach enables one to directly assess the impact of secretory trafficking on spike N-glycosylation using mass spectrometry. A critical role for conformational barriers in the maturation of N-glycans through mass spectrometry and MD simulations was elucidated. Cell culture and murine studies demonstrated that alterations in secretory trafficking and N-glycan remodeling had a minimal impact on the interactions of the spike protein with the immune system. Finally, single-particle cryo-electron microscopy reconstructions revealed the structural basis of antibody interactions among the various spike glycoforms.

At the fundamental level, this investigation establishes the secretory routing principles that control spike N-glycan maturation. It was shown that the maturation of unobstructed N-glycan sites such as at Asn1173 require only a few encounters with glycan-modifying enzymes and hence, are agnostic to the exact secretory route taken to reach the plasma membrane. In contrast, obstructed sites such as at Asn343, a modulator of RBD conformations (12, 15) and Asn616, which interacts with the fusion peptide-proximal region and the N-glycan at Asn657 (84), require recycling through ER-Golgi for efficient N-glycan processing. It has been shown previously that the deletion of the N-glycan at Asn657 dramatically enhances the infectivity of the SARS-CoV-2 pseudovirus particles in cell culture assays (84). The present invention demonstrates a structural role for N-glycans in protecting the fusion apparatus, likely to minimize premature viral interactions with host cells. Furthermore, the maturation outcomes of N-glycans at Asn343 and Asn616 during the secretory process may differ among various SARS-CoV-2 variants. This variability is attributed to evolutionary changes in the RBD conformational dynamics during the emergence of SARS-CoV-2 variants. For instance, it is likely that the exposure and maturation of the Asn343 N-glycan is enhanced by the Asp614Gly mutation, which favors the up-conformation of the RBDs (85). Nevertheless, the data collectively indicate that changes in N-glycosylation have a minimal impact on the structure, conformations, and interactions of the spike protein with the immune system. For instance, the structural and functional analyses of the spike-KxK construct suggest that the conformational dynamics and immunogenic conformations are established early during spike biogenesis, prior to the maturation of N-glycans. In conjunction with previous studies that investigated N-glycan deletions (12 15, 86), these findings highlight that the mere presence of an N-glycan, rather than its specific composition, serves as the primary determinant of spike conformations and immune interactions. This insight is particularly relevant in the context of spike-based genetic vaccines, as it underscores the potential to direct protein engineering efforts toward enhancing export properties and immunogenicity, with reduced concerns regarding N-glycosylation changes.

A major focus in the design of genetic vaccines during the COVID-19 pandemic was spike immunogenicity (87-89). This investigation highlights a strategy to balance the immunogenicity of a vaccine-encoded immunogen with minimal biogenesis burden on the host cells. For instance, the spike dibasic motif can potentially engage and divert the coatomer from the retrograde retrieval of cellular proteins such as ER-resident chaperones. This is consistent with the secretory leakage of ER-resident chaperones into the extra-cellular environment upon heterologous spike expression (90). Disruption of the coatomer housekeeping functions such as the maintenance of chaperone pools compromises secretory homeostasis and signaling, which are linked to auto-immune diseases, cancer, and various developmental disorders (36-40). The data on the spike-AxA and M-spike-AxA constructs suggest a way to avoid interference with coatomer housekeeping functions. It was shown that the spike-AxA and M-spike-AxA proteins are comparable with “wild-type” vaccine counterparts in binding to neutralizing antibodies in cell culture assays. Furthermore, when introduced as DNA plasmids in mice, the spike-AxA protein generates a superior neutralizing immune response relative to the “wild-type” vaccine counterpart. Finally, the single particle cryoEM reconstructions show structural similarity between the spike-AxA and spike-KxH constructs, the latter being equivalent to the “wild-type” vaccine counterpart.

These findings demonstrate that the RBD conformational states required for host interactions are established early during N-glycan processing, supporting the hypothesis that this facilitates SARS-CoV-2 in overcoming tissue-specific variations in N-glycan processing and broadening its tissue tropism during infection. Collectively, these structure-function data represent a substantial step forward in design principles that optimize immunogenicity while minimizing biogenesis burden in the cells of COVID-19 genetic vaccine recipients. More broadly, the coatomer-binding motif emerges as a conserved structural feature among the spike proteins of various human-infecting coronaviruses. This includes not only those associated with severe diseases, such as SARS-CoV, MERS-CoV, and SARS-CoV-2, but also endemic coronaviruses including HCoV-229E and HCoV-NL63, which contribute to 10-30% of upper respiratory tract infections and common colds (91). Inactivating this motif may present a broadly applicable strategy to mitigate the secretory interference associated with spike-based genetic vaccines targeting these diverse coronaviruses.

This approach could be crucial in reducing potential complications related to the export of spike proteins, particularly following repeated vaccinations that enhance the anti-viral immune response (92). Ultimately, this strategy contributes to the development of more effective immunization protocols against a range of CoVs. In the future, the widespread adoption of genetic vaccination technology will necessitate the establishment of design principles that account for variations in immunogen production, post-translational modifications, and export outcomes at the tissue levels and between population groups. This will be critical not only for protection against infectious pathogens but also against metabolic diseases, evidenced by recent promising clinical trials for pancreatic cancer (93).

The present investigation identifies several critical gaps in the understanding of the fundamental cell biology associated with COVID-19 genetic vaccines. Our findings indicate that the spike-KxK and M-spike-KxK constructs exhibit relatively limited interactions with antibodies in cell culture assays. This limitation is attributed to the His1271Lys substitution, which enhances interactions with coatomer, thereby restricting spike protein export to the cell surface (17, 49). Consequently, it was unexpected that the spike-KxK and M-spike-KxK constructs elicited a robust neutralizing response in mice. These observations suggest two potential hypotheses.

First, it is plausible that the spike-KxK and M-spike-KxK constructs undergo proteolytic processing, leading to the generation of peptides that are predominantly presented to the immune system, rather than intact spike proteins. If this hypothesis is validated, it would imply an enrichment of the immune response against linear epitopes, while the spike molecules exported to the cell surface would primarily contribute to the immune response against conformational epitopes. In this context, it is important to note that COVID-19 genetic vaccines developed by Moderna, Pfizer-BioNTech, and AstraZeneca encode a functional coatomer-binding motif (42), facilitating the generation of both retained and exported spike molecules. However, the distinct immunological fate of the retained spike molecules remains poorly understood. Peptide-dominated presentation may alter T-cell responses (94), and potentially contribute to the generation of non-neutralizing antibodies targeting epitopes that are not exposed in properly folded, functional spike proteins.

Alternatively, it is possible that the overexpression of the spike protein saturates cellular coatomer, resulting in the export of the spike protein to the cell surface. Should this hypothesis hold true, strategies aimed at enhancing the immune response through long-lived and self-amplifying genetic material (95, 96) may significantly disrupt normal secretory functions in the cells of vaccine recipients. Collectively, the present investigation underscores the urgent need for mechanistic investigations into the cell biology and immunology of spike-based genetic vaccines against present and emerging coronaviruses.

The investigation underscores the critical importance of comprehensively elucidating the interactions between the coatomer and viral proteins. Apart from the hijacking of the coatomer by the spike during coronavirus assembly (19, 41 97), previous studies have demonstrated that the coatomer is exploited by various other viruses at multiple stages of their infection cycle. For example, genetic studies have implicated the coatomer in viral entry processes, such as those of influenza virus, human papillomavirus 16, and SV40, as well as in the replication of poliovirus and hepatitis C virus (98-103). Moreover, a direct role for coatomer hijacking has been established in virus-mediated suppression of host immune responses. The E3/19K protein of adenoviruses, which cause the common cold, serves as a prime example. This type I membrane protein contains a cytosolic dibasic motif that directly interacts with the coatomer (28). This interaction is essential for E3/19K-mediated sequestration of the major histocompatibility complex class I (MHCI) in endomembrane compartments, thereby inhibiting immune activation (104, 105). Conversely, cowpox virus, which infects a range of mammalian species, employs an indirect mechanism to engage the coatomer and evade immune detection. Its CPXV203 protein features a Lys Thr Glu Leu (KTEL) (SEQ ID NO: 16) motif that interacts with the Lys Asp Glu Leu (KDEL) (SEQ ID NO: 17) receptor in endomembrane compartments while simultaneously binding to the MHCI complex (106, 107). As the KDEL receptor is an endogenous coatomer client (34, 108, 109), its engagement by CPXV203 facilitates the sequestration of the MHCI complex within the endomembrane system (110). Together with research on the hijacking of the coatomer by the coronavirus spike protein, these findings highlight the coatomer as a pivotal component in the molecular mechanisms of viral pathogenesis and immune evasion. This emerging role positions the coatomer as a promising target for advancing fundamental virological research such as in understanding stress caused by improper secretion of host proteins due to coatomer hijacking by viral proteins, and in the development of innovative therapeutic strategies.

In conclusion, this investigation provides structure-function insights into the control of SARS-CoV-2 spike protein biogenesis and immune interactions by secretory routing. This provides a path for a new generation of COVID-19 vaccines that minimally interfere with endogenous secretion and a fundamental and broadly applicable paradigm for the design of potentially safer genetic vaccines.

### REFERENCES

1. Uhlen, et al. (2019). Sci Signal 12.
2. Venne, et al. (2014). Proteomics 14:513-524.
3. Schjoldager, et al. (2020). Nat Rev Mol Cell Biol 21:729-749.
4. Stanley, et al. (2009). N-Glycans. In Essentials of Glycobiology, A. Varki, R. D. Cummings, J. D. Esko, H. H. Freeze, P. Stanley, C. R. Bertozzi, G. W. Hart, and M. E. Etzler, eds. (Cold Spring Harbor (NY)).
5. Lin, W. J., and Salton, S. R. (2013). Front Endocrinol (Lausanne) 4:96.
6. Reily, et al. (2019). Nat Rev Nephrol 15:346-366.
7. Bagdonaite, I., and Wandall, H. H. (2018). Glycobiology 28:443-467.
8. Frana, et al. (1985). J Virol 56:912-920.
9. Sturman, L., and Holmes, K. (1985). Trends in Biochemical Sciences 10:17-20.
10. Sturman, et al. (1985). J Virol 56:904-911.
11. Watanabe, et al. (2020). Site-specific glycan analysis of the SARS-CoV-2 spike. Science doi: 10.1126/science.abb9983.
12. Casalino, et al. (2020). ACS Cent Sci 6:1722-1734.
13. Casas-Sanchez, et al. (2022). mBio 13:e0371821.
14. Repp, et al. (1985). J Biol Chem 260:15873-15879.
15. Sztain, et al. (2021). Nat Chem 13:963-968.
16. Yang, et al. (2020). Elife 9.
17. Cattin-Ortole, et al. (2021). Nature Communications 12:5333.
18. Lontok, et al. (2004). J Virol 78:5913-5922.
19. McBride, et al. (2007). J Virol 81:2418-2428.
20. Corman, et al., (2018). Adv Virus Res 100:163-188.
21. Cui, et al. (2019). Nat Rev Microbiol 17:181-192.
22. Tang, et al. (2022). J Clin Virol 155:105246.
23. Cosson, P., and Letourneur, F. (1994). Science 263:1629-1631.
24. Faini, et al. (2012). Science 336:1451-1454.
25. Jackson, et al. (2012). Dev Cell 23:1255-1262.
26. Jackson, et al. (1990). EMBO J 9:3153-3162.
27. Jackson et al. (1993). J Cell Biol 121:317-333.
28. Nilsson, et al. (1989). Cell 58:707-718.
29. Beck, et al. (2009). FEBS Lett 583:2701-2709.
30. Dodonova, et al. (2015). Science 349:195-198.
31. Popoff, et al. (2011). Cold Spring Harb Perspect Biol 3:a005231.
32. Barre, et al. (2005). FEBS J 272:1063-1071.
33. Hamada, et al. (2004). Mol Cell Biol 24:8007-8017.
34. Majoul et al. (2001). Dev Cell 1:139-153.
35. Mitrovic, et al. (2008). Mol Biol Cell 19:1976-1990.
36. Steiner, et al. (2022). Nat Commun 13:2321.
37. Astroski, et al. (2021). Neurobiol Aging 101:57-69.
38. Dell'Angelica, E. C., and Bonifacino, J. S. (2019). Annu Rev Cell Dev Biol 35:131-168.
39. Pu, et al. (2018). Biomed Pharmacother 103:373-380.
40. Watkin, et al. (2015). Nat Genet 47:654-660.
41. Dey, et al. (2023). Nat Commun 14:8358.
42. Heinz, F. X., and Stiasny, K. (2021). NPJ Vaccines 6:104.
43. Dey, et al. (2022). Commun Biol 5:115.
44. Li, et al. (2023). Acta Pharm Sin B 13:3043-3053.
45. Ma, W., and Goldberg, J. (2013). EMBO J 32:926-937.
46. Wrapp, et al. (2020). Science 367:1260-1263.
47. Kwon, et al. (2018). Cell Rep 22:1798-1809.
48. Shi, et al. (2022). Structure 30:1233-1244 e1237.
49. Jennings, et al. (2021). FEBS Lett 595:1758-1767.
50. Remoroza, et al. (2021). J Proteome Res 20:4475-4486.
51. Casalino, et al. (2021). Int J High Perform Comput Appl 35:432-451.
52. Humphrey, et al. (1996). J Mol Graph 14:33-38:27-38.
53. Shrake, A., and Rupley, J. A. (1973). J Mol Biol 79:351-371.
54. Vallee, et al. (2000). J Biol Chem 275:41287-41298
55. Michaud-Agrawal, et al. (2011). J Comput Chem 32:2319-2327.
56. Corbett, et al. (2020). SARS-CoV-2 mRNA vaccine design enabled by prototype pathogen preparedness. Nature 586:567-571.
57. Liu, et al. (2020). Nature 584:450-456.
58. Wang, et al. (2021). Science 373.
59. Bepler, et al. (2019). Nat Methods 16:1153-1160.
60. Punjani, et al. (2017). Nat Methods 14:290-296.
61. Zivanov, et al. (2018). Elife 7.
62. Zhang, et al. (2021). Science 372:525-530.
63. Afonine, et al. (2018). Acta Crystallogr D Struct Biol 74:531-544.
64. Emsley, P., and Cowtan, K. (2004). Acta Crystallogr D Biol Crystallogr 60:2126-2132.
65. Pettersen, et al. (2004). J Comput Chem 25:1605-1612.
66. Cai, et al. (2020). Science 369:1586-1592.
67. Watanabe, et al. (2021). ACS Cent Sci 7:594-602.
68. Moremen, et al. (1994). Glycobiology 4:113-125.
69. Stanley, P. (2011). Cold Spring Harb Perspect Biol 3.
70. Henderson, et al. (2020). Glycans on the SARS-CoV-2 Spike Control the Receptor Binding Domain Conformation. bioRxiv.
71. Pang, et al. (2022). Commun Biol 5:1170.
72. Korber, et al. (2020). Cell 182:812-827 e819.
73. Nabel, et al. (2022). Science 375:eabl6251.
74. Pinto, et al. (2020). Nature 583:290-295.
75. Yuan, et al. (2020). Science 368:630-633.
76. Shi, et al. (2020). Nature 584:120-124.
77. Jones, et al. (2021). Sci Transl Med 13.
78. Westendorf, et al. (2022). Cell Rep 39:110812.
79. Zhou, et al. (2022). Science 376:eabn8897.
80. Cao, Y et al. (2022). Cell Rep 41:111845.
81. Starr, et al. (2021). Nature 597:97-102.
82. Pinto, et al. (2021). Science 373:1109-1116.
83. Dey et al. (2022). Commun Biol., 5:115.
84. Yang, et al. (2022). Sci Adv 8:eabq8678
85. Gobeil, et al. (2021). Cell Rep 34:108630.
86. Henderson, et al. (2021). Cell Rep 34:108630.
87. Kalnin et al. (2021). NPJ Vaccines 6: 61.
88. Polack, et al. (2020). N Engl J Med 383:2603-2615.
89. Walsh, et al. (2020). N Engl J Med 383:2439-2450.
90. Guo, et al. (2022). Sci Adv 8:eade5085.
91. Paules, et al. (2020). JAMA 323:707-708.
92. Declercq, J et al. (2024). Sci Transl Med 16:eadn2364.
93. Rojas, et al. (2023). Nature 618:144-150.
94. Karsten, et al. (2022). Clin Transl Immunology 11:e1410.
95. Bloom, et al. (2021). Gene Ther 28:117-129.
96. Tews, B. A., and Meyers, G. (2017). Methods Mol Biol 1499:15-35.
97. Youn, et al. (2005). J Virol 79:13209-13217.
98. Cherry, et al. (2006). PLoS Pathog 2: e102.
99. Konig, et al. (2010). Nature 463:813-817.
100. Laniosz, V et al. (2009). J Virol 83:8221-8232.
101. Richards, et al. (2002). Mol Biol Cell 13:1750-1764.
102. Tai, et al. (2009). Cell Host Microbe 5:298-307.
103. Thompson, J. A., and Brown, J. C. (2012). J Virol Antivir Res 1.
104. Burgert, H. G., and Kvist, S. (1985). Cell 41:987-997.
105. Cox, J et al. (1991). J Exp Med 174:1629-1637.
106. McCoy, et al. (2012). PLoS Biol 10:e1001432.
107. McCoy, et al. (2013). Mol Immunol 55:156-158.
108. Majoul, et al. (1998). J Cell Biol 143: 601-612.
109. Semenza, et al. (1990). Cell 61:1349-1357.
110. Byun et al. (2007). Cell Host Microbe 2:306-315.

What is claimed is:

1. An immunogenic construct, comprising:
a coronavirus spike protein with at least one substitution therein.

2. The immunogenic construct of claim 1, wherein the coronavirus spike protein comprises a substitution at one or both of the spike tail dibasic motif residues 1269Lys-Leu-His1271.

3. The immunogenic construct of claim 2, wherein the substitution is 1269Lys-Leu-Lys1271 or 1269Ala-Leu-Ala.

4. The immunogenic construct of claim 1, wherein the coronavirus spike protein comprises at least one sequence selected from the group consisting of GVALAYT (SEQ ID NO: 2), GVKLKYT (SEQ ID NO: 3), PHAVAVH (SEQ ID NO: 7), PHKVKVH (SEQ ID NO: 8), VEAIAIQ (SEQ ID NO: 9), VEKIKI (SEQ ID NO: 10), FEAVAVQ (SEQ ID NO: 11), and FEKVKVQ (SEQ ID NO: 12).

5. The immunogenic construct of claim 1, wherein the coronavirus is a severe acute respiratory coronavirus 2 (SARS-CoV-2), a Middle East respiratory syndrome coronavirus (MERS-CoV), a human coronavirus 229E (HCoV 229E), or a human coronavirus NL63 (HCoV NL63).

6. An immunological composition comprising the immunogenic construct of claim 1 and an immunologically acceptable excipient or adjuvant.

7. A method for eliciting an immune response against a coronavirus in a subject in need thereof, comprising:
administering to the subject an amount of the immunological composition of claim 6 effective to elicit a protective antibody response.

8. A plasmid, comprising:
a nucleic acid sequence that encodes an mRNA for a mutated coronavirus spike; and
a promoter operably linked to the nucleic acid sequence that regulates expression of the mRNA.

9. The plasmid of claim 8, wherein the mRNA encodes at least one protein with a sequence selected from the group consisting of SEQ ID NOS: 2, 3, 7, 8, 9, 10, 11, and 12.

10. The plasmid of claim 8, wherein the coronavirus is a severe acute respiratory coronavirus 2 (SARS-CoV-2), a Middle East respiratory syndrome coronavirus (MERS-CoV), a human coronavirus 229E (HCoV 229E), or a human coronavirus NL63 (HCoV NL63).

11. A genetic vaccine, comprising:
a mRNA that encodes a mutated spike protein, wherein said protein, when produced in a recipient of the vaccine, elicits a protective immune response against a coronavirus.

12. The genetic vaccine of claim 11, wherein the spike protein has a substitution at one or both of the spike tail dibasic motif residues 1269Lys-Leu-His1271.

13. The genetic vaccine of claim 12, wherein the substitution is 1269Lys-Leu-Lys1271 or 1269Ala-Leu-Ala.

14. The genetic vaccine of claim 11, wherein the mRNA encodes at least one spike protein sequence selected from the group consisting of GVALAYT (SEQ ID NO: 2), GVKLKYT (SEQ ID NO: 3), PHAVAVH (SEQ ID NO: 7), PHKVKVH (SEQ ID NO: 8), VEAIAIQ (SEQ ID NO: 9), VEKIKI (SEQ ID NO: 10), FEAVAVQ (SEQ ID NO: 11), and FEKVKVQ (SEQ ID NO: 12).

15. The genetic vaccine of claim 11, wherein the coronavirus is a severe acute respiratory coronavirus 2 (SARS-CoV-2), a Middle East respiratory syndrome coronavirus (MERS-CoV), a human coronavirus 229E (HCoV 229E), or a human coronavirus NL63 (HCoV NL63).

16. A method for preventing an onset of a coronaviral infection in a subject, comprising:
inoculating the subject with a first dose of the genetic vaccine of claim 11 effective to elicit anti-coronaviral antibody production in the subject.

17. The method of claim 16, further comprising administering to the subject at least a second dose of the genetic vaccine.

18. The method of claim 16, wherein the coronaviral infection is a severe acute respiratory coronavirus 2 infection.

19. A genetic vaccine directed against a severe acute respiratory coronavirus 2, comprising:
a mRNA that encodes a mutated spike protein with a sequence comprising GVALAYT (SEQ ID NO: 2) or GVKLKYT (SEQ ID NO: 3) or a combination thereof.


SEQUENCE LISTING (18 sequences)

SEQ ID NO: 1 - protein, 7 aa, synthetic construct
GVKLHYT

SEQ ID NO: 2 - protein, 7 aa, synthetic construct
GVALAYT

SEQ ID NO: 3 - protein, 7 aa, synthetic construct
GVKLKYT

SEQ ID NO: 4 - protein, 7 aa, synthetic construct
PHKVHVH

SEQ ID NO: 5 - protein, 7 aa, synthetic construct
VEKIHIQ

SEQ ID NO: 6 - protein, 7 aa, synthetic construct
FEKVHVQ

SEQ ID NO: 7 - protein, 7 aa, synthetic construct
PHAVAVH

SEQ ID NO: 8 - protein, 7 aa, synthetic construct
PHKVKVH

SEQ ID NO: 9 - protein, 7 aa, synthetic construct
VEAIAIQ

SEQ ID NO: 10 - protein, 7 aa, synthetic construct
VEKIKIQ

SEQ ID NO: 11 - protein, 7 aa, synthetic construct
FEAVAVQ

SEQ ID NO: 12 - protein, 7 aa, synthetic construct
FEKVKVQ

SEQ ID NO: 13 - protein, 5 aa, synthetic construct
SSHED

SEQ ID NO: 14 - protein, 5 aa, synthetic construct
ASHDD

SEQ ID NO: 15 - protein, 5 aa, synthetic construct
TSHDD

SEQ ID NO: 16 - protein, 5 aa, synthetic construct
SSAEA

SEQ ID NO: 17 - protein, 4 aa, Cowpox virus
KTEL

SEQ ID NO: 18 - protein, 4 aa, unidentified
KDEL